(19) United States
(12) Patent Application Publication
(10) Pub. No.: US 2025/0346853 A1
(43) Pub. Date: Nov. 13, 2025

(54) GENETICALLY-MODIFIED FILAMENTOUS FUNGI FOR PRODUCTION OF EXOGENOUS PROTEINS HAVING REDUCED OR NO N-LINKED GLYCOSYLATION

(71) Applicant: DYADIC INTERNATIONAL (USA), INC., Jupiter, FL (US)

(72) Inventors: Ronen Tchelet, Budapest (HU); Mark Aaron Emalfarb, Jupiter, FL (US); Mari Mäkinen, Espoo (FI); Markku Saloheimo, Espoo (FI)

(21) Appl. No.: 18/550,037

(22) PCT Filed: Mar. 10, 2022
(86) PCT No.: PCT/IB2022/052138

§ 371 Date: Sep. 11, 2023

Related U.S. Application Data
(60) Provisional application No. 63/159,492, filed on Mar. 11, 2021.

(51) Int. Cl. C12N 1/14; C12N 15/80; C12P 21/00; C12R 1/645

(52) U.S. Cl. CPC C12N 1/145; C12N 15/80; C12P 21/00; C12R 2001/645

(57) ABSTRACT

Ascomycetous filamentous fungi genetically modified to produce proteins having reduced or no N-glycans of mammalian proteins are provided, comprising deletion or disruption of stt3 and/or cwh8 genes.

### FIELD OF THE INVENTION

The present invention relates to genetically-modified ascomycetous filamentous fungi, having reduced expression and/or activity of STT3 and/or CWH8 proteins. The genetically modified filamentous fungi are used for robust production of recombinant proteins with partial or no-N-linked glycosylation.

### BACKGROUND OF THE INVENTION

The expression and purification of recombinant proteins having post-translational protein modifications, such as glycosylation or phosphorylation, can only be achieved using eukaryotic expression systems. Eukaryotic protein expression systems, including mammalian and insect cell lines, plant and fungi have become indispensable for the production of functional eukaryotic proteins.

As eukaryotic organisms, yeast and fungi are able to perform post-translational modifications, including N- and O-glycosylation, but protein glycosylation in yeast and fungi is different from that in mammalian cells. To overcome these problems, the possibility of reengineering the N-glycosylation pathway has been explored, especially in the species most frequently used for the production of heterologous proteins (e.g., Saccharomyces cerevisiae, Pichia pastoris, Yarrowia lipolytica, Hansenula polymorpha, and Aspergillus and Trichoderma species).

Parsaie Nasab et al., (2013, Appl Environ Microbiol., 79(3): 997-1007) describe a synthetic N-glycosylation pathway to produce recombinant proteins carrying human N-glycans in S. cerevisiae. The work by Parsaie Nasab et al. is also described in US 2011/0207214, which discloses cells modified to express lipid-linked oligosaccharide (LLO) flippase activity in the ER membrane. The flippase enables the flipping of LLO containing 1, 2 or 3 mannoses on the cytosolic side of the ER to the luminal side. The work is further reviewed along with other related studies in De Wachter et al., (2018, Engineering of Yeast Glycoprotein Expression. In: Advances in Biochemical Engineering/Biotechnology. Springer, Berlin, Heidelberg).

U.S. Pat. Nos. 7,029,872, 7,326,681, 7,629,163, and 7,981,660 disclose cell lines having genetically modified glycosylation pathways that allow them to carry out a sequence of enzymatic reactions, which mimic the processing of glycoproteins in humans. Eukaryotes such as unicellular and multicellular fungi, which ordinarily produce high-mannose-containing N-glycans, are modified to produce N-glycans such as Man₅GlcNAc₂ or other structures along human glycosylation pathways.

U.S. Pat. No. 9,359,628 discloses genetically engineered strains of Pichia capable of producing proteins with smaller glycans. In particular, the genetically engineered strains are capable of expressing either or both of an α-1,2-mannosidase and glucosidase II. The genetically engineered strains can be further modified such that the OCH1 gene is disrupted. Methods of producing glycoproteins with smaller glycans using such genetically engineered stains of Pichia are also provided.

U.S. Pat. Nos. 8,268,585 and 8,871,493 to the Applicant of the present invention disclose a transformation system in the field of filamentous fungal hosts for expressing and secreting heterologous proteins or polypeptides. Also disclosed is a process for producing large amounts of polypeptides or proteins in an economical manner. The system comprises a transformed or transfected fungal strain of the genus Chrysosporium, more particularly of Chrysosporium lucknowense and mutants or derivatives thereof. Also disclosed are transformants containing Chrysosporium coding sequences, as well expressing-regulating sequences of Chrysosporium genes.

Thermothelomyces heterothallica (Th. heterothallica) strain C1 (recently renamed from Myceliophthora thermophila, which was renamed from Chrysosporium lucknowense) is a thermo-tolerant ascomycetous filamentous fungus producing high levels of cellulases, which made it attractive for production of these and other enzymes on a commercial scale.

Wild type C1 was deposited in accordance with the Budapest Treaty with the number VKM F-3500 D, deposit date Aug. 29, 1996. High Cellulase (HC) and Low Cellulase (LC) strains have also been deposited, as described, for example, in U.S. Pat. No. 8,268,585.

U.S. Pat. No. 9,695,454 discloses compositions including filamentous fungal cells, such as Trichoderma fungal cells, having reduced protease activity and expressing fucosylation pathway. Further described are methods for producing a glycoprotein having fucosylated N-glycan, using genetically modified filamentous fungal cells, for example, Trichoderma fungal cells, as the expression system.

U.S. Pat. Nos. 7,449,308 and 7,935,513 disclose eukaryotic host cells having modified oligosaccharides which may be modified further by heterologous expression of a set of glycosyltransferases, sugar transporters and mannosidases to become host-strains for the production of mammalian, e.g., human therapeutic glycoproteins. N-glycans made in the engineered host cells have a Man₅GlcNAc₂ core structure which may then be modified further by heterologous expression of one or more enzymes, e.g., glycosyltransferases, sugar transporters and mannosidases, to yield human-like glycoproteins.

U.S. Pat. Nos. 8,268,585 and 8,871,493 disclose a transformation system in the field of filamentous fungal hosts for expressing and secreting heterologous proteins or polypeptides. Also disclosed is a process for producing large amounts of polypeptide or protein in an economical manner. The system comprises a transformed or transfected fungal strain of the genus Chrysosporium, more particularly of Chrysosporium lucknowense and mutants or derivatives thereof. Also disclosed are transformants containing Chrysosporium coding sequences, as well expression-regulating sequences of Chrysosporium genes.

U.S. Pat. No. 9,175,296 discloses a fungal host strain of Chrysosporium lucknowense. Also disclosed is a method for homologous and/or heterologous production of a pure protein with a purity of higher than 75%, a method for production of artificial protein mixes and a method for simplified screening of strains functionally expressing a desired enzyme. U.S. Pat. No. 9,175,296 further discloses an isolated promoter sequence suitable for the transcriptional control of gene expression in Chrysosporium lucknowense (recently re-named Thermothelomyces heterothallica) and a method for isolating a fungal host strain of Chrysosporium lucknowense wherein the protease secretion is less than 20% of the protease secretion of Chrysosporium lucknowense strain UV 18-25.

There is a need for an expression system for producing recombinant proteins that is able to produce high yields of proteins with partial or no-N-linked glycosylation, such that the proteins are suitable for a variety of industrial and pharmaceutical usages.

### SUMMARY OF THE INVENTION

The present invention provides genetically modified ascomycetous filamentous fungi genetically modified to produce proteins having reduced or no N-glycan modifications of mammalian proteins. In particular, the present invention provides Thermothelomyces heterothallica strain C1 as an exemplary ascomycetous filamentous fungus genetically modified to produce recombinant proteins having reduced or no N-glycan modifications. In some embodiments, the fungi disclosed herein were modified to be deficient of stt3 and/or cwh8 genes.

The present invention is based in part on the finding that Th. heterothallica genetically-modified as disclosed herein produces proteins having reduced or no glycans compared to the non-modified strain. This is in contrast to hitherto described expression systems, which produce proteins with large variation in the obtained N-glycans.

Advantageously, the modified ascomycetous filamentous fungi cells of the present invention enable the production of heterologous proteins having partial post-translational modifications. These proteins may be used in a variety of applications in which the fully glycosylated proteins are not suitable for. For example, the proteins can be designed for desired solubility and/or biological activity. The proteins having reduced amounts of N-glycan may show reduced immunogenicity compared the fully glycosylated proteins. The partially N-glycosylated proteins of the invention may be used as a primary material for additional or other protein modifications. In addition, the non-N-glycosylated proteins may be used as potential control proteins for various glycosylated forms and mixtures in pharmacokinetic/pharmacodynamic studies. Furthermore, the partial glycosylated proteins described herein may have different therapeutic effects compared to the natively glycosylated proteins, since the therapeutic effects, are often depend on N-glycosylation.

Advantageously, the modified ascomycetous filamentous fungi cells of the present invention produce proteins with high yield and stability. The protein levels obtained using the Th. heterothallica cells of the present invention are much higher than those obtained using mammalian cells, such as CHO cells, or yeasts.

The present invention therefore provides an efficient system for producing eukaryotic recombinant proteins with reduced or no N-glycans, suitable for a variety of usages in the pharmaceutical and non-pharmaceutical industries.

According to one aspect, the present invention provides a genetically modified ascomycetous filamentous fungus capable of producing a protein of interest with reduced or no N-linked glycosylation, the genetically modified filamentous fungus comprising at least one cell having reduced expression and/or activity of STT3 and/or CWH8.

According to some embodiments, the at least one cell comprising at least one exogenous polynucleotide encoding the protein of interest.

According to some embodiments, the at least one cell has a reduced expression and/or activity of STT3.

According to some embodiments, the STT3 comprises an amino acid sequence having at least 75%, or at least 80%, or at least 85%, or at least 90%, or at least 95%, or at least 99%, or 100% identity to the amino acid of Thermothelomyces heterothallica STT3. According to certain embodiments, the Thermothelomyces heterothallica STT3 comprises the amino acid of SEQ ID NO: 27.

According to some embodiments, the at least one cell has a reduced expression and/or activity of CWH8.

According to some embodiments, the CWH8 comprises an amino acid sequence having at least 75%, or at least 80%, or at least 85%, or at least 90%, or at least 95%, or at least 99%, or 100% identity to the amino acid of Thermothelomyces heterothallica CWH8. According to certain embodiments, the Thermothelomyces heterothallica CWH8 comprises the amino acid of SEQ ID NO: 28.

According to some embodiments, the modified filamentous fungus comprises at least one cell having reduced expression and/or activity of STT3 and CWH8 proteins.

According to some embodiments, the genetic modification comprises deletion or disruption of the stt3 gene. According to some embodiments, the genetic modification comprises deletion or disruption of the stt3 gene such that the modified filamentous fungus produces reduced amount of a catalytic subunit of the oligosaccharyltransferase (OST) complex. According to some embodiments, the genetic modification comprises deletion or disruption of the stt3 gene such that the modified filamentous fungus fails to produce a catalytic subunit of the oligosaccharyltransferase (OST) complex.

According to some embodiments, the genetic modification comprises deletion or disruption of the cwh8 gene. According to some embodiments, the genetic modification comprises deletion or disruption of the cwh8 gene such that the modified filamentous fungus produces reduced amount of a functional dolichyl pyrophosphate phosphatase. According to some embodiments, the genetic modification comprises deletion or disruption of the cwh8 gene such that the modified filamentous fungus fails to produce a functional dolichyl pyrophosphate phosphatase.

According to some embodiments, the modified filamentous fungus express proteins with reduced amount of N-linked glycosylation. According to certain embodiments, the modified filamentous fungus expresses proteins having less than 20% N-linked glycosylation compared to non-modified fungus. According to additional embodiments, the modified filamentous fungus express proteins without N-linked glycosylation.

According to some embodiments, the ascomycetous filamentous fungus is of a genus within the group Pezizomycotina.

According to some embodiments, the ascomycetous filamentous fungus is of a genus selected from the group consisting of Thermothelomyces, Myceliophthora, Trichoderma, Aspergillus, Penicillium, Rasamsonia, Chrysosporium, Corynascus, Fusarium, Neurospora, and Talaromyces.

According to some embodiments, the ascomycetous filamentous fungus is of a species selected from the group consisting of Thermothelomyces heterothallica (also denoted Myceliophthora thermophila), Myceliophthora lutea, Aspergillus nidulans, Aspergillus funiculosus Aspergillus niger, Aspergillus oryzae, Trichoderma reesei, Trichoderma harzianum, Trichoderma longibrachiatum, Trichoderma viride, Rasamsonia emersonii. Penicillium chrysogenum, Penicillium verrucosum, Sporotrichum thermophile, Corynascus fumimontanus, Corynascus thermophilus, Chrysosporium lucknowense, Fusarium graminearum, Fusarium venenatum, Neurospora crassa, and Talaromyces piniphilus.

According to some embodiments, the ascomycetous filamentous fungus is a Thermothelomyces heterothallica strain comprising rDNA sequence having at least 95%, or at least 96%, or at least 97%, or at least 98%, or at least 99% or 100% identity to the nucleic acid sequence set forth in SEQ ID NO: 29.

According to some embodiments, the ascomycetous filamentous fungus is Thermothelomyces heterothallica. According to some embodiments, the ascomycetous filamentous fungus is Thermothelomyces heterothallica C1.

In some embodiments, the C1 is a strain selected from the group consisting of: W1L#100I (prt-Δalp1Δchi1Δalp2Δpyr5) deposit no. CBS141153, UV18-100f (prt-Δalp1, Δpyr5) deposit no. CBS141147, W1L#100I (prt-Δalp1Δchi1Δpyr5) deposit no. CBS141149, and UV18-100f (prt-Δalp1Δpep4Δalp2Δprt1Δpyr5) deposit no. CBS141143 and derivatives thereof. Each possibility represents a separate embodiment of the present invention.

According to some embodiments, the protein of interest is selected from the group consisting of an enzyme, structural protein, vaccine antigen and components thereof.

According to some embodiments, the protein of interest is a secreted protein. According to certain embodiments, the protein of interest has a leader peptide. According to other embodiments, the protein of interest is an intracellular protein. In certain embodiments, the intracellular protein is a membrane or vesicle bound protein.

According to some embodiments, the protein of interest is an antibody or a fragment thereof. According to certain embodiments, the antibody is IgG4 or IgG1. According to additional embodiments, the antibody is a bi- or multiple specific antibody.

According to some embodiments, the protein of interest is a therapeutic protein.

According to some embodiments, the protein of interest is a vaccine protein antigen.

The polynucleotide encoding the protein of interest may form part of a DNA construct or expression vector.

According to some embodiments, the at least one exogenous polynucleotide is a DNA construct or an expression vector further comprising at least one regulatory element operable in said ascomycetous filamentous fungus. According to certain embodiments, the regulatory element is selected from the group consisting of a regulatory element endogenous to said fungus and a regulatory element heterologous to said fungus.

According to some embodiments, the genetically modified ascomycetous filamentous fungus is designed to produce secreted proteins.

In some embodiments, the ascomycetous filamentous fungus according to the present invention is genetically modified to express an antibody.

In some embodiments, the ascomycetous filamentous fungus is a strain further modified to delete one or more genes encoding an endogenous protease.

According to some embodiments, the genetically modified ascomycetous filamentous fungus comprises at least one cell having reduced expression and/or activity of at least 5, 6, 7, 8, 9, 10, 11, 12, 13, or 14 proteases. Each possibility represents a separate embodiment of the invention. According to certain embodiments, the modified filamentous fungus comprises at least one cell having reduced expression and/or activity of at least 9, 10, 11, 12, 13, or 14 proteases. Each possibility represents a separate embodiment of the invention.

According to another aspect, the present invention provides a method for generating an ascomycetous filamentous fungus that is capable of producing proteins with reduced or no N-linked glycosylation, comprising:
(a) reducing the expression and/or activity of STT3 protein of the ascomycetous filamentous fungus; and/or
(b) reducing the expression and/or activity of CHW8 protein of the ascomycetous filamentous fungus.

According to some embodiments, the method comprising:
(a) deleting or disrupting the stt3 gene of the ascomycetous filamentous fungus as to reduce the production of a functional catalytic subunit of the oligosaccharyltransferase (OST) complex; and/or
(b) deleting or disrupting the chw8 gene of the ascomycetous filamentous fungus as to reduce the production of a functional dolichyl pyrophosphate phosphatase.

According to some embodiments, the fungus fails to produce a functional catalytic subunit of the oligosaccharyltransferase (OST) complex.

According to some embodiments, the fungus fails to produce a functional dolichyl pyrophosphate phosphatase.

According to some embodiments, the fungus fails to produce a functional catalytic subunit of the oligosaccharyltransferase (OST) complex and a functional dolichyl pyrophosphate phosphatase.

According to some embodiments, the method further comprises introducing into the ascomycetous filamentous fungus an exogenous polynucleotide encoding a heterologous protein of interest thereby expressing the heterologous protein of interest having reduced or no N-glycans in the fungus.

According to another aspect, the present invention provides a method for producing a heterologous protein having reduced or no N-glycan modifications, the method comprising:
(i) providing an ascomycetous filamentous fungus genetically modified comprising at least one cell having reduced expression and/or activity of STT3 and/or CWH8 as described herein and at least one exogenous polynucleotide encoding a protein of interest according to the present invention;
(ii) culturing the ascomycetous filamentous fungus under conditions suitable for expressing the protein; and
(iii) recovering the protein.

In some embodiments, the protein is a heterologous mammalian protein recombinantly expressed in the ascomycetous filamentous fungus. In some particular embodiments, the protein is a human protein recombinantly expressed in the ascomycetous filamentous fungus. In other embodiments, the protein is a protein of a companion and/or farm animal recombinantly expressed in the ascomycetous filamentous fungus.

According to a further aspect, the present invention provides a recombinant protein produced by the ascomycetous filamentous fungus genetically modified according to the present invention.

In some embodiments, the recombinant protein produced by the ascomycetous filamentous fungus genetically modified according to the present invention is a pharmaceutical grade protein.

According to a further aspect, the present invention provides a method of producing at least one protein of interest, the method comprising culturing the genetically modified fungus as described herein in a suitable medium; and recovering the at least one protein product.

According to some embodiments, the recovering step comprises recovering the protein from the growth medium, from the fungal mass or both.

According to some embodiments, the protein is recovered from the growth medium. According to certain embodiment, at least 50%, 60%, 70%, 80%, 90% or 95% of the protein is secreted.

It is to be understood that any combination of each of the aspects and the embodiments disclosed herein is explicitly encompassed within the disclosure of the present invention.

These and further aspects and features of the present invention will become apparent from the detailed description, examples and claims which follow.

### BRIEF DESCRIPTION OF THE FIGURES

FIG. 1. Lipid-linked oligosaccharide biosynthesis pathway and transfer of the oligosaccharide to a nascent polypeptide at the membrane of the endoplasmic reticulum (ER) in eukaryotic cells.

FIGS. 2A-2B. N-glycan patterns and abundance of different glycan forms on native proteins of fungi produced in a bioreactor in a non-glycomodified C1 strain (2A) and in the stt3 deletion strain M3210 (2B).

FIGS. 3A-3B. N-glycan patterns and abundance of different glycan forms on native proteins of fungi produced in a bioreactor in a non-glycomodified C1 strain (3A) and in the cwh8 deletion strain M3211 (3B).

FIGS. 4A-4B. N-glycan patterns and abundance of different glycan forms on a monoclonal antibody produced in a non-glycomodified C1 strain (4A) and in the stt3 deletion strain M3480 (4B).

FIGS. 5A-5B. N-glycan patterns and abundance of different glycan forms on a monoclonal antibody produced in a non-glycomodified C1 strain (5A) and in the cwh8 deletion strain M3481 (5B).

### DETAILED DESCRIPTION OF THE INVENTION

The present invention provides alternative, highly efficient system for producing proteins having reduced or no N-linked glycosylation. The system of the invention is based in part on the filamentous fungus Thermothelomyces heterothallica C1 and particular strains thereof, which have been previously developed as a natural biological factory for protein as well as secondary metabolite production. The present invention in some embodiments provides genetically modified fungi having reduced expression and/or activity of STT3 and/or CWH8 proteins. The genetically modified fungi in some embodiments have reduced or abolished expression and/or activity of multiple proteases.

The proteins produced by genetically-modified fungus as described herein are suitable for a variety of pharmaceutical and non-pharmaceutical applications.

According to one aspect, the present invention provides a genetically modified filamentous fungus to produce a protein of interest, the genetically modified filamentous fungus comprises at least one cell having reduced expression and/or activity of STT3 and/or CWH8 proteins.

According to an additional aspect, the present invention provides a genetically modified filamentous fungus capable of producing recombinant proteins having reduced or no N-linked glycosylation, wherein the genetic modification comprises:
(i) deletion or disruption of the stt3 gene such that the genetically modified filamentous fungus fails to produce a catalytic subunit of the oligosaccharyltransferase (OST) complex;
(ii) deletion or disruption of the cwh8 gene such that the genetically modified filamentous fungus fails to produce a functional dolichyl pyrophosphate phosphatase; or
(iii) deletion or disruption of both stt3 and cwh8 genes.

The term “disruption” means that a gene can be structurally disrupted so as to comprise at least one mutation or structural alteration such that the disrupted gene is incapable of directing the efficient expression of a full-length fully functional gene product. The term “disruption” also encompasses that the disrupted gene or one of its products can be functionally inhibited or inactivated such that a gene is either not expressed or is incapable of efficiently expressing a full-length and/or fully functional gene product. Functional inhibition or inactivation can result from a structural disruption and/or interruption of expression at either level of transcription or translation. The term “disruption” also encompasses attenuation or knocking down of the gene expression.

Protein glycosylation, namely, the covalent attachment of oligosaccharides to side chains of newly synthesized polypeptide chains in cells, is an ordered process in eukaryotic cells involving a series of enzymes that sequentially add and remove saccharide moieties. N-glycosylation is the process in which an oligosaccharide is attached to the side chain of an asparagine residue, particularly an asparagine which occurs in the sequence Asn-Xaa-Ser/Thr, where Xaa represents any amino acid except Pro.

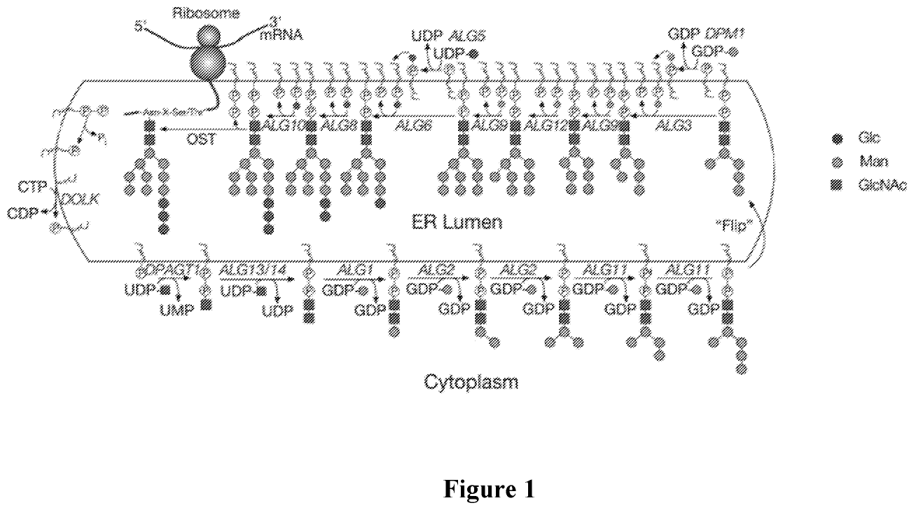

N-glycosylation initiates in the endoplasmic reticulum (ER), where the oligosaccharide Glc₃Man₉GlcNAC₂ is assembled on a lipid carrier, dolichol-pyrophosphate, and subsequently transferred to selected asparagine residues of polypeptides that have entered the lumen of the ER. FIG. 1 illustrates the biosynthesis pathway of the lipid-linked oligosaccharide and the transfer of the oligosaccharide to a nascent polypeptide at the membrane of the ER in eukaryotic cells. The biosynthesis of the lipid-linked oligosaccharide requires the activity of several specific glycosyltransferases. It begins at the cytoplasmic side of the ER membrane and terminates in the lumen where oligosaccharyltransferase (OST) selects N-X-S/T sequons of a nascent polypeptide and generates the N-glycosidic linkage between the side chain amide of asparagine and the oligosaccharide. The flipping of the lipid-linked oligosaccharide from outside the ER to the inside is carried out by a flippase located at the ER membrane. Following transfer to the nascent polypeptide, the oligosaccharide is typically trimmed by glucosidases and mannosidases and the nascent glycoprotein is then transferred to the Golgi apparatus for further processing.

The synthesis of the dolichol-pyrophosphate-bound oligosaccharide is essentially conserved in all known eukaryotes. However, further processing of the oligosaccharide as the glycoprotein moves along the secretory pathway varies greatly between lower eukaryotes such as fungi or yeasts and higher eukaryotes such as animals and plants. Thus, the final composition of a sugar side chain is different between various organisms, and depends upon the host.

In microorganisms such as yeasts, typically additional mannose and/or mannosylphosphate sugars are added, resulting in “hypermannosylated” type N-glycans which may contain up to 30-50 mannose residues.

In animal cells, including human, companion animal and other mammalian cells, the nascent glycoprotein is transferred to the Golgi apparatus where mannose residues are removed by Golgi-specific 1,2-mannosidases. Processing continues as the protein proceeds through the Golgi by a number of modifying enzymes including N-acetylglucosamine transferases (GnT I, GnT II, GnT III, GnT IV, GnT V, GnT VI), mannosidase II and fucosyltransferases that add and remove specific sugar residues. Finally, the N-glycans are acted on by galactosyl transferases (GalT) and sialyltransferases (ST) and the finished glycoprotein is released from the Golgi apparatus. The N-glycans of animal glycoproteins have bi-, tri-, or tetra-antennary structures, and may typically include galactose, mannose, fucose and N-acetylglucosamine. Commonly the terminal residues of the N-glycans consist of sialic acid.

Th. heterothallica, unlike yeast, does not have hypermannosylated N-glycans, but rather has “oligo mannose” glycans—Man₃ to Man₈₋₉—and hybrid type glycans containing both Man and HexNAc residues (Man₃HexNac-Man₈HexNac). The exact structure of these hybrid glycans is not completely known. The hybrid glycans have the typical mannose residues but in addition an unknown HexNAc attached via a yet uncharacterized bond.

The present invention is directed to genetic modification of the N-glycosylation pathway such that it produces reduced amount of N-glycans.

As used herein, “glycan” refers to an oligosaccharide chain that can be linked to a carrier such as an amino acid, peptide, polypeptide, lipid or a reducing end conjugate. The present invention particularly relates to N-linked glycans (“N-glycan”) conjugated to a polypeptide N-glycosylation site such as -Asn-Xaa-Ser/Thr- by N-linkage to side-chain amide nitrogen of asparagine residue (Asn), where Xaa is any amino acid residue except Pro. The present invention may further relate to glycans as part of dolichol-phospho-oligosaccharide (Dol-P-P-OS) precursor lipid structures, which are precursors of N-linked glycans in the endoplasmic reticulum of eukaryotic cells. The precursor oligosaccharides are bound by their reducing end to two phosphate residues on the dolichol lipid.

The term “stt3 gene” refers to the gene encoding Dolichyl-diphosphooligosaccharide-protein glycosyltransferase subunit. It is the catalytic subunit of the oligosaccharyltransferase (OST) complex that catalyzes the initial transfer of a defined glycan (Glc3Man9GlcNAc2 in eukaryotes) from the lipid carrier dolichol-pyrophosphate to an asparagine residue within an Asn-X-Ser/Thr consensus motif in nascent polypeptide chains, the first step in protein N-glycosylation. STT3 protein catalyzes the reaction:

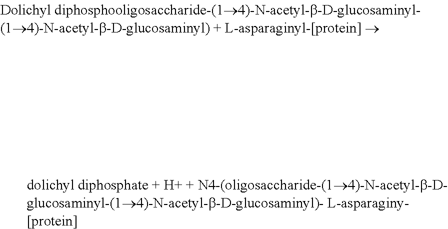

The genetically modified ascomycetous filamentous fungi of the present invention is genetically modified by deletion or disruption of the stt3 gene such that the fungi fail to produce a functional catalytic subunit of the oligosaccharyltransferase (OST) complex. The genetically modified ascomycetous filamentous fungi of the present invention does not display a detectable oligosaccharyltransferase (OST) activity.

The term “cwh8 gene” refers to the gene encoding dolichyldiphosphatase. CWH8 catalyzes the reaction:

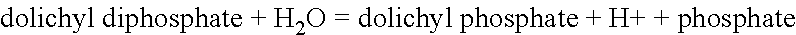

The genetically modified ascomycetous filamentous fungi of the present invention is genetically modified by deletion or disruption of the cwh8 gene such that the fungi fail to produce a functional dolichyldiphosphatase. The genetically modified ascomycetous filamentous fungi of the present invention does not display a detectable dolichyldiphosphatase activity.

Ascomycetous filamentous fungi as defined herein refer to any fungal strain belonging to the group Pezizomycotina. The Pezizomycotina comprises, but is not limited to the following groups:
Sordariales, including genera:
   Thermothelomyces (including species: heterothallica and thermophila),
   Myceliophthora (including the species lutea and unnamed species),
   Corynascus (including the species fumimontanus),
   Neurospora (including the species crassa);
Hypocreales, including genera:
   Fusarium (including the species graminearum and venenatum),
   Trichoderma (including the species reesei, harzianum, longibrachiatum and viride);
Onygenales, including genera:
   Chrysosporium (including the species lucknowense);
Eurotiales, including genera:
   Rasamsonia (including the species emersonii),
   Penicillium (including the species verrucosum),
   Aspergillus (including the species funiculosus, nidulans, niger and oryzae)
   Talaromyces (including the species piniphilus (formerly Penicillium funiculosum).

It is to be understood that the above list is not conclusive, and is meant to provide an incomplete list of industrially relevant filamentous ascomycetous fungal species.

While there may be filamentous ascomycetous species outside Pezizomycotina, that group does not contain Saccharomycotina, which contains most commonly known non-filamentous industrially relevant genera, such as Saccharomyces, Komagataella (including formerly Pichia pastoris), Kluyveromyces or Taphrinomycotina, which contains some other commonly known non-filamentous industrially relevant genera, such as Schizosaccharomyces.

All taxonomical categories above are defined according to the NCBI Taxonomy browser (ncbi.nlm.nih.gov/taxonomy) as of the date of the patent application.

It must be appreciated that fungal taxonomy is in constant move, and the naming and the hierarchical position of taxa may change in the future. However, a skilled person in the art will be able to unambiguously determine if a particular fungal strain belongs to the group as defined above.

According to certain embodiments, the filamentous fungus genus is selected from the group consisting of Myceliophthora, Thermothelomyces, Aspergillus, Penicillium, Trichoderma, Rasamsonia, Chrysosporium, Corynascus, Fusarium, Neurospora, Talaromyces and the like. According to some embodiments, the fungus is selected from the group consisting of Myceliophthora thermophila, Thermothelomyces thermophila (formerly M. thermophila), Thermothelomyces heterothallica (formerly M. thermophila and heterothallica), Myceliophthora lutea, Aspergillus nidulans, Aspergillus funiculosus Aspergillus niger, Aspergillus oryzae, Penicillium chrysogenum, Penicillium verrucosum, Trichoderma reesei, Trichoderma harzianum, Trichoderma longibrachiatum, Trichoderma viride, Chrysosporium lucknowense, Rasamsonia emersonii, Sporotrichum thermophile, Corynascus fumimontanus, Corynascus thermophilus, Fusarium graminearum, Fusarium venenatum, Neurospora crassa, and Talaromyces piniphilus.

In particular, the present invention provides Thermothelomyces heterothallica strain C1 as model for an ascomycetous filamentous fungus, capable of producing high amounts of stable proteins.

The terms “Thermothelomyces” and its species “Thermothelomyces heterothallica and thermophila” are used herein in the broadest scope as is known in the art. Description of the genus and its species can be found, for example, in Marin-Felix Y (2015. Mycologica 107(3): 619-632 doi.org/10.3852/14-228) and van den Brink J et al. (2012, Fungal Diversity 52(1): 197-207). As used herein “C1” or “Thermothelomyces heterothallica C1” or Th. heterothallica C1, or C1 all refer to Thermothelomyces heterothallica strain C1.

It is noted that the above authors (Marin-Felix et al., 2015) proposed splitting of the genus Myceliophthora based on differences in optimal growth temperature, morphology of the conidiospore, and details of the sexual reproduction cycle. According to the proposed criteria C1 clearly belongs to the newly established genus Thermothelomyces, which contain former thermotolerant Myceliophthora species rather than to the genus Myceliophthora, which remains to include the non-thermotolerant species. As C1 can form ascospores with some other Thermothelomyces (formerly Myceliophthora) strains with opposite mating type, C1 is best classified as Th. heterothallica strain C1, rather than Th. thermophila C1.

It must also be appreciated that the fungal taxonomy was also in constant change in the past, so the current names listed above may be preceded by a variety of older names beyond Myceliophthora thermophila (van Oorschot, 1977. Persoonia 9(3):403), which are now considered synonyms. For example, Thermothelomyces heterothallica (Marin-Felix et al., 2015. Mycologica, 3:619-63), is synonymized with Corynascus heterotchallicus, Thielavia heterothallica, Chrysosporium lucknowense and thermophile as well as Sporotrichium thermophile (Alpinis 1963. Nova Hedwigia 5:74).

It is further to be explicitly understood that the present invention encompasses any strain containing a ribosomal DNA (rDNA) sequence that shows 99% homology or more to Sequence No: 29, and all those strains are considered to be conspecific with Thermothelomyces heterothallica.

Particularly, the term Th. heterothallica strain C1 encompasses genetically modified sub-strains derived from the wild type strain, which have been mutated, using random or directed approaches, for example, using UV mutagenesis, or by deleting one or more endogenous genes. For example, the C1 strain may refer to a wild type strain modified to delete one or more genes encoding an endogenous protease. For example, C1 strains which are encompassed by the present invention include strain UV18-25, deposit No. VKM F-3631 D; strain NG7C-19, deposit No. VKM F-3633 D; and strain UV13-6, deposit No. VKM F-3632 D. Further C1 strain that may be used according to the teachings of the present invention include HC strain UV18-100f deposit No. CBS141147; HC strain UV18-100f deposit No. CBS141143; LC strain W1L#100I deposit No. CBS141153; and LC strain W1L#100I deposit No. CBS141149 and derivatives thereof.

It is to be explicitly understood that the teachings of the present invention encompass mutants, derivatives, progeny, and clones of the Th. heterothallica C1 strains, as long as these derivatives, progeny, and clones, when genetically modified according to the teachings of the present invention are capable of producing at least one protein product according to the teachings of the invention. As used herein, the term “progeny” refers to an unmodified or partially modified descendant from the parent fungal line, such as cell from cell. The term “parent strain” refers to a corresponding fungal strain not having reduced expression or activity of specific protease according to the invention.

Several Th. heterothallica C1 strains developed by the Applicant of the present invention are less sensitive to feedback repression by glucose and other fermentable sugars present in the growth medium as carbon source than conventional yeast strains and also most other ascomycetous filamentous fungal hosts, and consequently can tolerate higher feeding rate of the carbon source, leading to high yields production by this fungus.

According to some embodiments, the fungi growth medium comprises a carbon source selected from the group consisting of glucose, sucrose, xylose, arabinose, galactose, fructose, lactose, cellobiose, glycerol and any combination thereof.

The present invention is particularly directed to engineering reduced or no N-glycosylation modifications. It is noted that O-glycans may be present or removed or altered by further genetic modifications of the fungus.

The terms “reduced expression” or “inhibited expression” of a protein as described herein are used interchangeably and include, but are not limited to, deleting or disrupting the gene that encodes for the protein.

The terms “reduced activity” or “inhibited activity” of a protein as described herein are used interchangeably and include, but are not limited to, posttranslational modifications resulting in reduced or abolished activity of the protein.

It is to be understood that the genetic modifications according to the present invention are such that the genetically-modified fungus is able to grow at sufficient rates suitable for its intended use.

The above terms also encompass genetically modified sub-strains derived from the wild type strain, which have been mutated, using random or directed approaches, for example, using UV mutagenesis, or by deleting one or more endogenous genes.

Th. heterothallica fungi in general and strain C1 in particular show higher biomass production compared to yeast strains when grown in suitable conditions. Th. heterothallica fungi can grow in large volumes of 3 dimensions (3D) liquid cultures as well as on solid medium. Several strains developed by the Applicant of the present invention are less sensitive to feedback repression by glucose and other fermentable sugars present in the fungal growth medium as carbon source compared to conventional yeast and other fungi, and can tolerate high feeding rate of the carbon source leading to high yields. Furthermore, some of these strains provide significantly reduced medium viscosity when grown in commercial fermenters compared to the high viscosity obtained with non-glucose repressed wild type Th. heterothallica fungi or with other filamentous fungi known to be used for proteins production. The low viscosity may be attributed to the morphological change of the strain from having long and highly interlaced hyphae in the parental strain(s) to short and less interlaced hyphae in the developed strain(s). Low medium viscosity is highly advantageous in large scale industrial production in fermenters. For example, the Th. heterothallica C1 strain UV18-25, deposit No. VKM F-3631 D, which shows reduced sensitivity to glucose repression, has been grown industrially to produce recombinant enzymes at volumes of more than 100,000 liters.

The term “heterologous”, when referring to a gene, enzyme, protein or peptide sequence is used herein to describe a gene, enzyme, protein or peptide sequence that is not naturally found or expressed in ascomycetous filamentous fungi.

The term “endogenous”, when referring to a gene, enzyme, protein or peptide sequence such as a subcellular localization signal, refers to a gene, enzyme, protein or peptide sequence that is naturally present in the ascomycetous filamentous fungi.

The term “exogenous”, when referring to a polynucleotide, is used herein to describe a synthetic polynucleotide that is exogenously introduced into the ascomycetous filamentous fungi via transformation. The exogenous polynucleotide may be introduced into the ascomycetous filamentous fungi in a stable or transient manner, so as to produce a ribonucleic acid (RNA) molecule and subsequently a polypeptide molecule.

### Expression Vectors

According to some embodiments, the genetically modified ascomycetous filamentous fungus described herein comprising at least one exogenous polynucleotide encoding the protein of interest.

The polynucleotide encoding the protein of interest may form part of a DNA construct or expression vector.

The terms “expression construct”, “DNA construct” or “expression cassette” are used herein interchangeably and refer to an artificially assembled or isolated nucleic acid molecule which includes a nucleic acid sequence encoding a protein of interest and which is assembled such that the protein of interest is expressed in a target host cell. An expression construct typically comprises appropriate regulatory sequences operably linked to the nucleic acid sequence encoding the protein of interest. An expression construct may further include a nucleic acid sequence encoding a selection marker.

The terms “nucleic acid sequence”, “nucleotide sequence” and “polynucleotide” are used herein to refer to polymers of deoxyribonucleotides (DNA), ribonucleotides (RNA), and modified forms thereof in the form of a separate fragment or as a component of a larger construct. A nucleic acid sequence may be a coding sequence, i.e., a sequence that encodes for an end product in the cell, such as a protein. A nucleic acid sequence may also be a regulatory sequence, such as, for example, a promoter.

The terms “peptide”, “polypeptide” and “protein” are used herein to refer to a polymer of amino acid residues. The term “peptide” typically indicates an amino acid sequence consisting of 2 to 50 amino acids, while “protein” indicates an amino acid sequence consisting of more than 50 amino acid residues.

A sequence (such as a nucleic acid sequence and an amino acid sequence) that is “homologous” to a reference sequence refers herein to percent identity between the sequences, where the percent identity is at least 75%, preferably at least 80%, at least 85%, at least 90%, at least 95%, at least 98% or at least 99%. Each possibility represents a separate embodiment of the present invention. Homologs of the sequences described herein are encompassed within the present invention. Protein homologs are encompassed as long as they maintain the activity of the original protein. Homologous nucleic acid sequences include variations related to codon usage and degeneration of the genetic code. Sequence identity may be determined using nucleotide/amino acid sequence comparison algorithms, as known in the art.

Nucleic acid sequences encoding the protein of interest may be optimized for expression. Examples of such sequence modifications include, but are not limited to, an altered G/C content to more closely approach that typically found in ascomycetous filamentous fungi, and the removal of codons atypically found in the fungus, commonly referred to as codon optimization.

The phrase “codon optimization” refers to the selection of appropriate DNA nucleotides for use within a structural gene or fragment thereof that approaches codon usage within the organism of interest, and/or to a process of modifying a nucleic acid sequence for enhanced expression in the host cell of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in protein synthesis. Accordingly, genes can be tailored for optimal gene expression in a given organism based on codon optimization. Therefore, an optimized gene or nucleic acid sequence refers to a gene in which the nucleotide sequence of a native or naturally occurring gene has been modified in order to utilize statistically-preferred or statistically-favored codons within the organism.

The term “regulatory sequences” refer to DNA sequences which control the expression (transcription) of coding sequences, for example, promoters and terminators.

The term “promoter” is directed to a regulatory DNA sequence which controls or directs the transcription of another DNA sequence in vivo or in vitro. Usually, the promoter is located in the 5′ region (that is, precedes, located upstream) of the transcribed sequence. Promoters may be derived in their entirety from a native source, or be composed of different elements derived from different promoters found in nature, or even comprise synthetic nucleotide segments. Promoters can be constitutive (i.e. promoter activation is not regulated by an inducing agent and hence rate of transcription is constant), or inducible (i.e., promoter activation is regulated by an inducing agent). In most cases the exact boundaries of regulatory sequences have not been completely defined, and in some cases cannot be completely defined, and thus DNA sequences of some variation may have identical promoter activity.

The term “terminator” is directed to another regulatory DNA sequence which regulates transcription termination. A terminator sequence is operably linked to the 3′ terminus of the nucleic acid sequence to be transcribed.

According to some embodiment, the filamentous fungus is Th. heterothallica and the protein of interest is expressed in a construct having regulatory elements of Th. heterothallica. According to specific embodiments, the construct expressing the protein of interest comprises Th. heterothallica promoter and/or Th. heterothallica terminator.

The terms “Th. heterothallica promoter” and “Th. heterothallica terminator” indicate promoter and terminator sequences suitable for use in Th. heterothallica, i.e., capable of directing gene expression in Th. heterothallica. In some particular embodiments, C1 promoters and C1 terminators are used, which indicate promoter and terminator sequences capable of directing gene expression in C1.

According to some embodiments, the Th. heterothallica promoter/terminator is derived from an endogenous gene of Th. heterothallica. According to other embodiments the Th. heterothallica promoter/terminator is derived from a gene exogenous to Th. heterothallica.

Suitable constitutive promoters and terminators include, for example, those of C1 glycolytic genes such as phosphoglycerate kinase gene (PGK) (Uniprot: G2QLD8, NCBI Reference Sequence: XM_003665967), glyceraldehyde 3-phosphate dehydrogenase (GPD) (Uniprot: G2QPQ8, NCBI Reference Sequence: XM_003666768), phosphofructokinase (PFK) (Uniprot: G2Q605, NCBI Reference Sequence: XM_003659879); or the β-glucosidase 1 gene bgl1 (Accession number: XM_003662656); or triose phosphate isomerase (TPI) (Uniprot: G2QBR0, NCBI Reference Sequence: XM_003663200); or actin (ACT) (Uniprot: G2Q7Q5, NCBI Reference Sequence: XM_003662111); or the C1 cbh1 promoter (GenBank AX284115) or C1 chi1 promoter (GenBank HI550986). Additional promoters that can be used are Aspergillus nidulans gpdA promoter; and synthetic promoters described in Rantasalo et al. (2018 NAR 46(18):e111). As exemplary terminators, the terminator of the C1 chitinase 1 gene chi1 (GenBank HI550986), cellobiohydrolase 1 cbh1 (GenBank AX284115) can be used, or the yeast adh1 terminator.

The term “operably linked” means that a selected nucleic acid sequence is in proximity with a regulatory element (promoter or terminator) to allow the regulatory element to regulate expression of the selected nucleic acid sequence.

Expression constructs according to some embodiments of the present invention comprise a Th. heterothallica promoter sequence and a Th. heterothallica terminator sequence operably linked to a nucleic acid sequence encoding a protein. In some particular embodiments, expression constructs of the present invention comprise a C1 promoter sequence and a C1 terminator sequences operably linked to a nucleic acid sequence encoding an enzyme.

A particular expression construct may be assembled by a variety of different methods, including conventional molecular biology methods such as polymerase chain reaction (PCR), restriction endonuclease digestion, in vitro and in vivo assembly methods, as well as gene synthesis methods, or a combination thereof. Exemplary expression constructs and methods for their construction are provided in the Examples section below.

### Deletion of stt3 and/or cwh8 Genes

Gene deletion techniques enable the partial or complete removal of a gene, thereby eliminating its expression. In such methods, deletion of the gene may be accomplished by homologous recombination using a plasmid that has been constructed to contiguously contain the 5′ and 3′ regions flanking the gene.

Gene deletion may also be performed by inserting into the gene a disruptive nucleic acid construct, also termed herein a deletion construct. A disruptive construct may be simply a selectable marker gene accompanied by 5′ and 3′ regions homologous to the gene. The selectable marker enables identification of transformants containing the disrupted gene. Alternatively or additionally, the disruptive nucleic acid construct may comprise one or more polynucleotides encoding heterologous proteins to be expressed in the host cell.

Exemplary deletion constructs for stt3 and cwh8 and procedures for carrying out the deletion are provided in the Examples section below. As described herein, the stt3 and cwh8 genes are deleted using a disruptive construct comprising a selectable marker, as shown in Example 1 below.

The deletion(s) may be confirmed using PCR with appropriate primers flanking the disruptive construct(s).

### Genetically-Engineered Th. heterothallica

Th. heterothallica cells genetically engineered to produce proteins having reduced or no N-linked glycans according to the present invention are generated by modifying, such as deleting, at least one of the two endogenous genes of Th. heterothallica, stt3 and cwh8, such that the genes fail to produce functional proteins.

The deletion of the endogenous genes is described above and is also demonstrated in the Examples section below.

According to yet another aspect, the present invention provides a method of producing an exogenous protein, the method comprising culturing the genetically modified fungus, particularly Th. heterothallica C1 fungi of the present invention in a suitable medium; and recovering the protein products.

According to certain embodiments, the medium comprises a carbon source selected from the group consisting of glucose, sucrose, xylose, arabinose, galactose, fructose, lactose, cellobiose, and glycerol. According to certain embodiments the carbon source is waste obtained from ethanol production or other bioproduction from starch, sugar beet and sugar cane such as molasses comprising fermentable sugars, starch, lignocellulosic biomass comprising polymeric carbohydrates such as cellulose and hemicellulose.

According to some embodiment, the exogenous protein is purified from the fungal growth medium.

According to other embodiments, the exogenous protein is extracted from the fungal mass. Any method as is known in the art for extracting and purifying proteins from vegetative tissues can be used.

According to a further aspect, the present invention provides an exogenous protein produced by the genetically modified fungus, particularly the genetically modified Th. heterothallica C1 of the present invention.

The expression of an exogenous polynucleotide is carried out by introducing into fungal cells, particularly into the nucleus, an expression construct comprising a nucleic acid encoding a protein to be expressed in the fungi. In particular, the genetic modification according to the present invention means incorporation of the expression construct to the host genome.

Introduction of an expression construct into fungal cells, i.e., transformation of fungi, can be performed by methods as known in the art, for example, using the protoplast transformation method described in the Examples section below.

To facilitate easy selection of transformed cells, a selection marker may be transformed into the fungal cells. A “selection marker” indicates a polynucleotide encoding a gene product conferring a specific type of phenotype that is not present in non-transformed cells, such as an antibiotic resistance (resistance markers), ability to utilize a certain resource (utilization/auxotrophic markers) or expression of a reporter protein that can be detected, e.g. by spectral measurements. Auxotrophic markers are typically preferred as a means of selection in the food or pharmaceutical industry. The selection marker can be on a separate polynucleotide co-transformed with the expression construct, or on the same polynucleotide of the expression construct. Following transformation, positive transformants are selected by culturing the cells on e.g., selective media according to the chosen selection marker. In some cases, a split marker system is used, where the selection marker is split into two plasmids and a functional selection marker is formed only when the two plasmids are co-transformed and joined together via homologous recombination.

### List of Sequences

| | SEQ | |
|---|---|---|
| | ID | |
| Element | NO: | Description/Remarks |
| 5′ arm of | 1 | |
| the stt3 | | |
| deletion | | |
| construct | | |
| 3′ arm of | 2 | |
| the stt3 | | |
| deletion | | |
| construct | | |
| 5′ arm of | 3 | |
| the cwh8 | | |
| deletion | | |
| construct | | |
| 3′ arm of | 4 | |
| the cwh8 | | |
| deletion | | |
| construct | | |
| Primer | 5 | For verifying integration of the stt3 deletion |
| | | construct at the 5′ end of the gene. |
| Primer | 6 | For verifying integration of the stt3 deletion |
| | | construct or cwh8 deletion construct at the 5′ end of |
| | | the gene. |
| Primer | 7 | For verifying integration of the stt3 deletion |
| | | construct at the 3′ end of the gene. |
| Primer | 8 | For verifying integration of the stt3 deletion |
| | | construct or cwh8 deletion construct at the 3′ end of |
| | | the gene. |
| Primer | 9 | For verifying complete deletion of the stt3 gene |
| Primer | 10 | For verifying complete deletion of the stt3 gene |
| Primer | 11 | For verifying complete deletion of the stt3 gene |
| Primer | 12 | For verifying complete deletion of the stt3 gene |
| Primer | 13 | For verifying integration of the cwh8 deletion |
| | | construct at the 5′ end of the gene. |
| Primer | 14 | For verifying integration of the cwh8 deletion |
| | | construct at the 3′ end of the gene. |
| Primer | 15 | For verifying complete deletion of the cwh8 gene |
| Primer | 16 | For verifying complete deletion of the cwh8 gene |
| Primer | 17 | For verifying complete deletion of the cwh8 gene |
| Primer | 18 | For verifying complete deletion of the cwh8 gene |
| 5′arm | 19 | |
| of the | | |
| Nivolumab | | |
| expression | | |
| construct | | |
| 3′ arm | 20 | |
| of the | | |
| Nivolumab | | |
| expression | | |
| construct | | |
| Primer | 21 | For verifying integration of the Nivolumab |
| | | expression cassette at the 5′ end of the cbhl gene |
| Primer | 22 | For verifying integration of the Nivolumab |
| | | expression cassette at the 5′ end of the cbhl gene |
| Primer | 23 | For verifying integration of the Nivolumab |
| | | expression cassette at the 3′ end of the cbhl gene |
| Primer | 24 | For verifying integration of the Nivolumab |
| | | expression cassette at the 3′ end of the cbhl gene |
| Primer | 25 | For verifying deletion of the cbhl gene |
| Primer | 26 | For verifying deletion of the cbhl gene |

List of C1 strains generated and used in this work*

| Name | Description |
|---|---|
| DNL132 | C1 |
| M3210 | C1 having deletion of stt3 |
| M3211 | C1 having deletion of cwh8 |
| M3480 | C1 having deletion of stt3 and expression of |
| | Nivolumab |
| M3481 | C1 having deletion of cwh8 and expression of |
| | Nivolumab |

*DNL132 was generated prior to this work

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

### Example 1—Deletion of C1 stt3 and cwh8 Genes

In order to reduce or eliminate N-glycosylation of secreted proteins in C1, the genes encoding the dolichyl-diphospho-oligosaccharide protein glycosyltransferase catalytic subunit STT3 and the dolichyldiphosphatase CWH8 were deleted. STT3 is a subunit of the multimeric oligosaccharyltransferase (OST) complex and is essential for the catalytic activity of the complex (Zufferey et al. 1995, EMBO Journal 14, 4949-4960). OST complex catalyzes the transfer of the oligosaccharide from a lipid carrier dolichylpyrophosphate to the selected asparagine residues of polypeptide chains. Dolichylpyrophosphate phosphatase CWH8 is postulated to be involved in recycling of the lipid carrier dolichylpyrophosphate. CWH8 is nonessential for the survival of cells but deletion of the corresponding gene results in N-glycosylation defect (van Berkel et al. 1999, Glycobiology 9, 243-253).

The deletions were carried out by transforming stt3 and cwh8 deletion constructs individually (one deletion/strain) into C1. The DNA constructs for deleting stt3 or cwh8 were constructed into two separate plasmids. The 5′ arm plasmid contained the stt3/cwh8 5′ flanking region fragment for integration and the first half of the pyr4 marker gene. The 3′ arm plasmid contained the second half of the pyr4 marker and the stt3/cwh8 3′ flanking region fragment for integration. The pyr4 marker fragments in these two plasmids overlap with each other. During transformation of the two plasmids to C1, the overlapping region undergoes homologous recombination between the plasmids at the same time as the 5′ and 3′ flanking region fragments recombine with genomic DNA on both sides of the gene to be deleted. Recombination between the selection marker fragments is mandatory for the marker gene to be functional and therefore enables the transformants to grow under selection. Approximately 500 bp from the end of the 5′ flanking region was added after the second half of the PYR4 marker in order to enable looping out the marker gene, if necessary. The different fragments of stt3 and cwh8 5′ and 3′ arm vectors were amplified from C1 genomic DNA and cloned with the marker genes into a backbone vector (pRS426) by yeast recombinational cloning (Colot et al. 2006, PNAS 103, 10352-10357).

The 5′ arm of stt3 deletion construct is set forth in SEQ ID NO: 1. The 5′ flank sequence corresponds to positions 1-930 of SEQ ID NO: 1 and the first half of the pyr4 marker gene corresponds to positions 938-2,717 of SEQ ID NO: 1. The 3′ arm of the stt3 deletion construct is set forth in SEQ ID NO: 2. The second half of the pyr4 marker gene corresponds to positions 1-1,257 of SEQ ID NO: 2. The direct repeat sequence corresponds to positions 1,266-1,777 of SEQ ID NO: 2. The 3′ flanking sequence corresponds to positions 1,785-2,715 of SEQ ID NO: 2.

The 5′ arm of cwh8 deletion construct is set forth in SEQ ID NO: 3. The 5′ flank sequence corresponds to positions 1-1,000 of SEQ ID NO: 3 and the first half of the pyr4 marker gene corresponds to positions 1,008-2,787 of SEQ ID NO: 3. The 3′ arm of cwh8 deletion construct is set forth in SEQ ID NO: 4. The second half of the pyr4 marker gene corresponds to positions 1-1,257 of SEQ ID NO: 4. The direct repeat sequence corresponds to positions 1,266-1,765 of SEQ ID NO: 4. The 3′ flanking sequence corresponds to positions 1,774-2,973 of SEQ ID NO: 4.

Both arms of the stt3/cwh8 deletion construct were excised from the plasmid backbones and transformed simultaneously into a C1 strain DNL132 having 9 deletions of protease genes. A pair of one 5′ arm vector and one 3′ arm vector was used in each transformation.

The transformant colonies growing on the selection medium plates were cultivated as streaks on the selective medium. Identification of transformants with correct integration of the deletion construct was carried out by PCR. Mycelium from the transformant streaks was dissolved in 20 mM NaOH and incubated at 100° C. to lyse the cells. 1-2 μl of this lysate was used as template for PCR with Phire Plant PCR kit™ (Thermo Fisher). The oligonucleotide primers used are shown in Table 1.

Integration of the deletion construct into the stt3 locus was demonstrated by two PCR reactions. Integration at the 5′ end of the gene was verified by a reaction with the primers set forth as SEQ ID NO: 5 and SEQ ID NO: 6. Amplification of a 1152 bp fragment indicated successful integration to stt3 locus at the 5′ end of the gene. Integration at the 3′ end of stt3 was verified with the primers set forth as SEQ ID NO: 7 and SEQ ID NO: 8. Amplification of a 1752 bp fragment indicated successful integration to stt3 locus at the 3′ end of the gene. Transformants positive for integration to the stt3 locus were further analysed by quantitative PCR with the primers set forth as SEQ ID NO: 9 and SEQ ID NO: 10 and with the primers set forth as SEQ ID NO: 11 and SEQ ID NO: 12 to demonstrate that the stt3 gene had been completely deleted from them. The transformant C1 strain, positive for integration of the construct into stt3 locus and negative for the presence of stt3 gene, was stored at −80° C. and given the strain number M3210.

The integration of the deletion construct into the cwh8 locus was demonstrated by two PCR reactions. Integration at the 5′ end of the gene was verified by a reaction with the primers set forth as SEQ ID NO: 13 and SEQ ID NO: 6. Amplification of a 1243 bp fragment indicated successful integration to cwh8 locus at the 5′ end of the gene. Integration at the 3′ end of the gene was verified with the primers set forth as SEQ ID NO: 14 and SEQ ID NO: 8. Amplification of a 2009 bp fragment indicated successful integration to cwh8 locus at the 3′ end of the gene. Transformants positive for integration to the cwh8 locus were further analysed by quantitative PCR with the primers set forth as SEQ ID NO: 15 and SEQ ID NO: 16 and with the primers set forth as SEQ ID NO: 17 and SEQ ID NO: 18 to demonstrate that the cwh8 gene had been completely deleted from them. The transformant C1 strain, positive for integration of the construct into cwh8 locus and negative for the presence of cwh8 gene, was stored at −80° C. and given the strain number M3211.

TABLE 1
Oligonucleotide primers for screening of stt3
and cwh8 deletions

| SEQ | |
|---|---|
| ID NO: | Sequence |
| 5 | CCGTCAAGGTACGAGAAACC |
| 6 | AGTTTGACAGTGCCCAGAGC |
| 7 | TTCTTAACACCGAGGGTTGG |
| 8 | AGCCTGGAAGGCCTATCTGG |
| 9 | ACAACAACCTCTTCCCTCCT |
| 10 | TCGTGAAAGCCTCCTCCA |
| 11 | ACAACGAAGCCATTGCCA |
| 12 | CAACCATGTAGCCGTAGGAG |
| 13 | GTTTCGGTTTTTGAGAACGG |
| 14 | CGTCCTAAAGCGTCCAATCG |
| 15 | GGTGACGTATCTGATGAGGG |
| 16 | CACAAATCTTCCTCGACCAC |
| 17 | GTGGTCGAGGAAGATTTGTG |
| 18 | TCACAAATGGTTTCCCACCT |

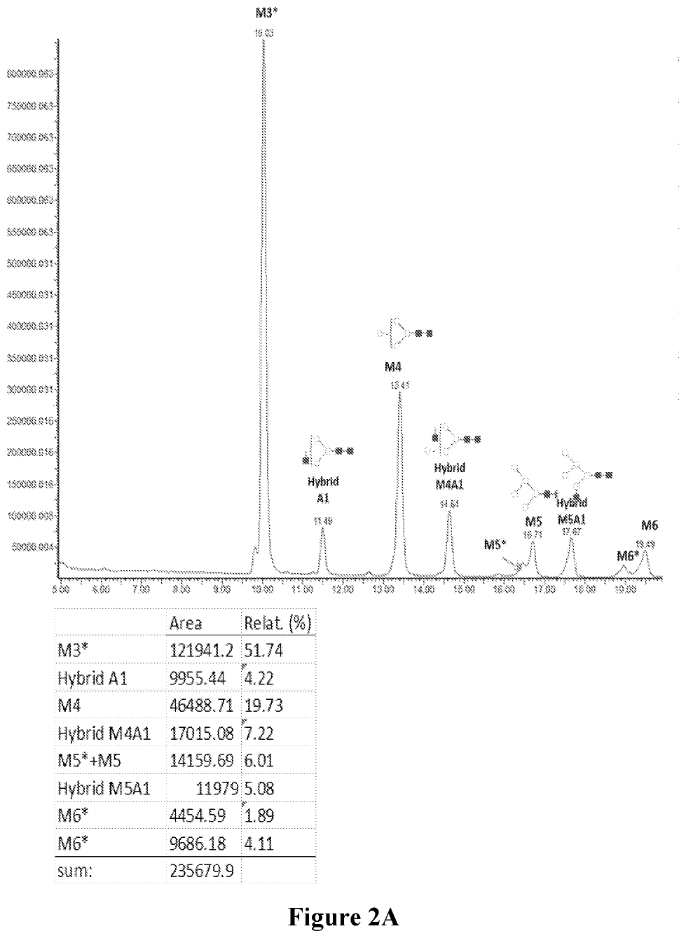

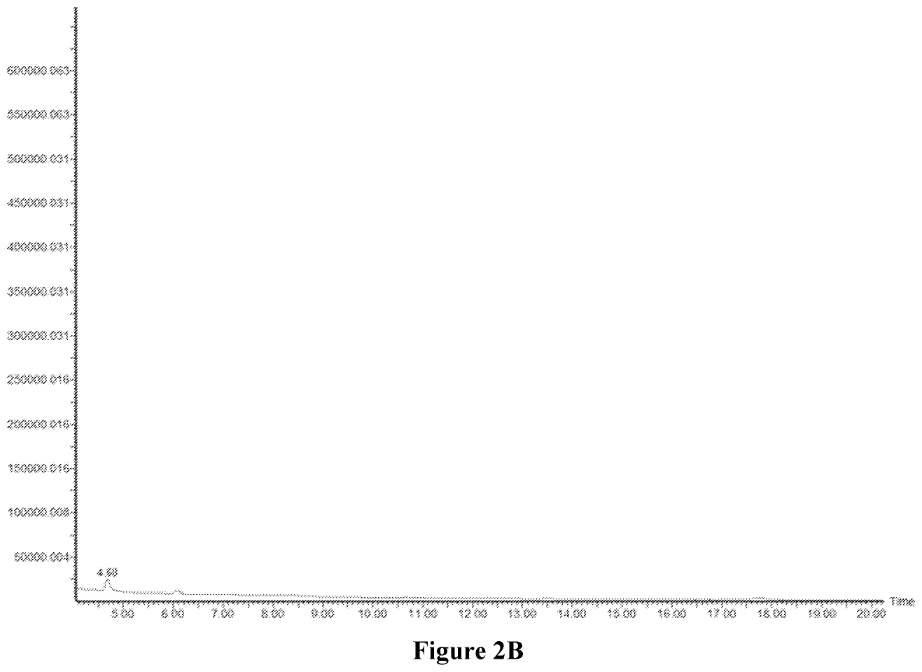

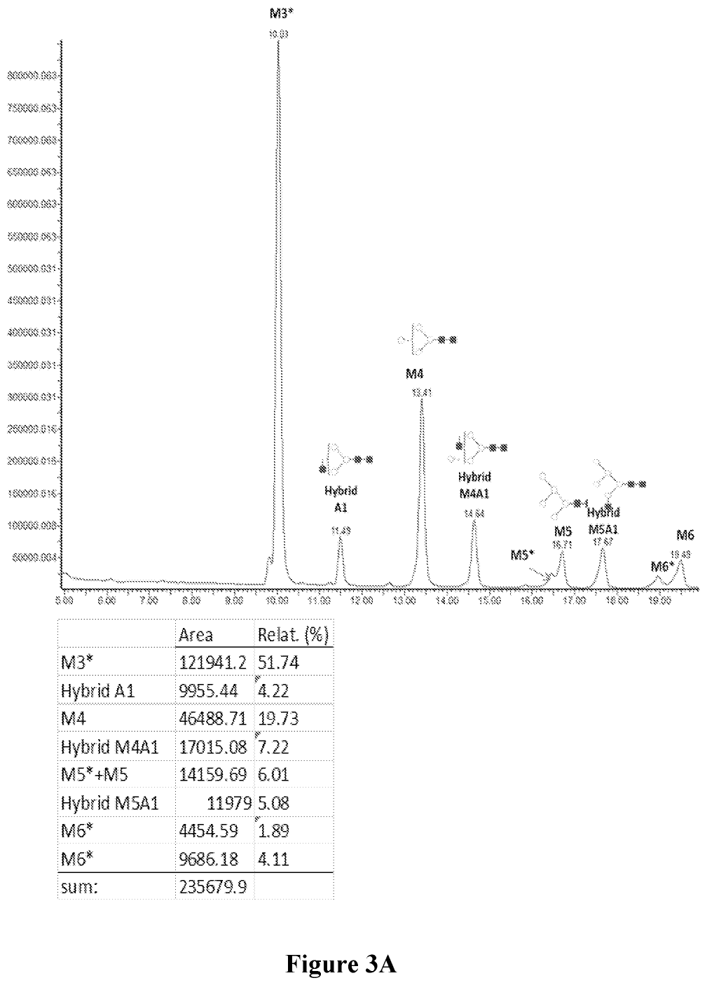

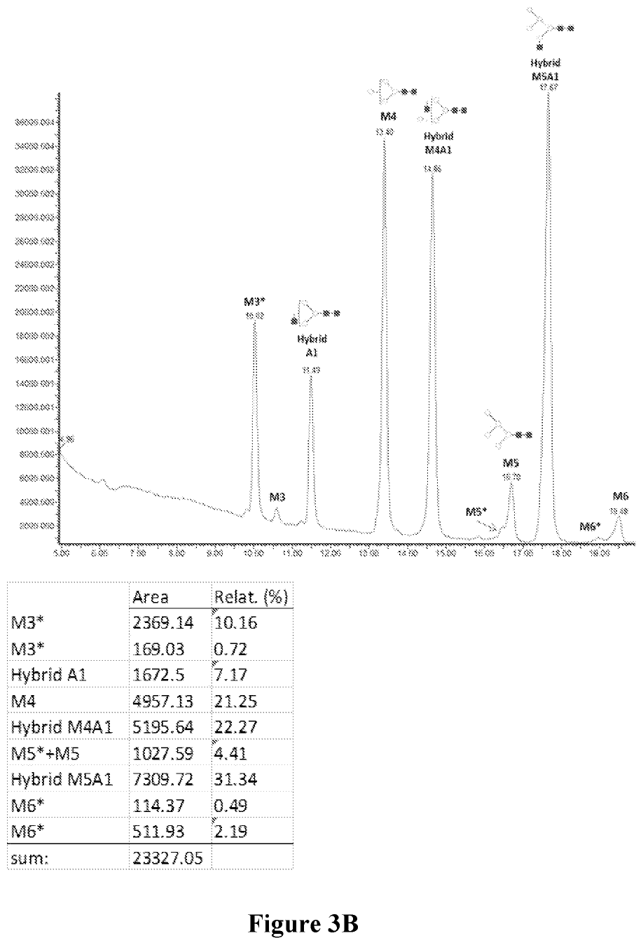

M3210 and M3211 strains were cultivated in 1 L bioreactors in fed-batch process in a medium containing yeast extract and glucose as the carbon source for 7 days. Supernatant samples from day 7 were centrifuged 3 times 13 500 rpm, 15 min and glycan analysis from total protein present in the supernatants was performed with the GlycoWorks™ RapiFluor-MS™ N-Glycan Kit (Waters) according to manufacturer's protocols. Strain M2864 with 9 protease deletions was used as a control in N-glycan analysis. No N-glycans could be detected from the culture supernatant of the stt3 deletion strain whereas the control strain showed a normal C1 glycan pattern (FIGS. 2A and 2B). Amount of N-glycans detected from the culture supernatant of the cwh8 deletion strain was approximately 10% of the N-glycan amount of the control strain (FIGS. 3A and 3B).

### Example 2—Production of a Monoclonal Antibody in Strains M3210 and M3211

In order to demonstrate production of a monoclonal antibody with no N-glycosylation and with reduced N-glycosylation, the heavy and light chain of the therapeutic antibody Nivolumab were expressed in the M3210 and M3211 strains having stt3 or cwh8 deletions, respectively.

The expression cassette was constructed in two parts into two separate plasmids as explained in Example 1. The 5′ arm of the construct contained the cbh1 5′ flanking region fragment for integration, an expression cassette where the Nivolumab light chain gene fused to the C1 CBH1 signal sequence is between bgl8 promoter and bgl8 terminator, nia1 marker gene and the first ½ of the hygromycin marker gene. The 3′ arm of the construct contained the last ½ of the hygromycin marker, a direct repeat sequence from the bgl8 terminator, an expression cassette where the Nivolumab heavy chain gene fused to a CBH1 signal sequence from C1 is between bgl8 promoter and chi1 terminator, and the cbh1 3′ flanking region fragment for integration.

The 5′ arm of the Nivolumab expression construct is set forth in SEQ ID NO: 19. The cbh1 5′ flank sequence corresponds to positions 1-1,957 of SEQ ID NO: 19. The bgl8 promoter sequence corresponds to positions 1,966-3,357 of SEQ ID NO: 19. The sequence encoding light chain fused to C1 CBH1 signal sequence corresponds to positions 3,358-4,053 of SEQ ID NO: 19, where positions 3,358-3,408 encode the CBH1 signal sequence and positions 3,409-4,053 encode the light chain. This sequence was obtained by codon-optimizing the human light chain gene for C1, and synthesis by Genscript. The synthetized sequence contained 40 bp flanks for bgl8 promoter and terminator. The bgl8 terminator sequence corresponds to positions 4,054-4,520 of SEQ ID NO: 19. The nia1 marker gene corresponds to positions 4,537-8,651 of SEQ ID NO: 19. The first ½ of the hygromycin marker gene corresponds to positions 8,660-10,360 of SEQ ID NO: 19. The fragments described above and the backbone vector pRS426 were assembled together by Gibson assembly to get the 5′ arm vector.

The 3′ arm of the Nivolumab expression construct is set forth in SEQ ID NO: 20. The ½ of the hygromycin marker gene corresponds to positions 1-1,732 of SEQ ID NO: 20. The chi1 terminator sequence corresponds to positions 2,082-2,727 of SEQ ID NO: 20. The sequence encoding heavy chain fused to C1 CBH1 signal sequence corresponds to positions 2,736-4,109 of SEQ ID NO: 20 where positions 4,059-4,109 encode the CBH1 signal sequence and positions 2,736-4,058 encode the heavy chain. This sequence was obtained by codon-optimizing the human heavy chain gene for C1, and synthesis by Genscript. It includes 40 bp flanks for bgl8 promoter and chi1 terminator. The bgl8 promoter sequence corresponds to positions 4,110-5,501 of SEQ ID NO: 20. The 3′ flank sequence corresponds to positions 5,510-6,266 of SEQ ID NO: 20. The fragments described above and the backbone vector pRS426 were assembled together by Gibson assembly to get the 3′ arm vector.

Transformation of the Nivolumab expression plasmids into M3210 and M3211 strains carrying stt3 or cwh8 deletions, respectively, and selection of transformants were done on selective medium plates containing 50 mg/l hygromycin. The transformants were screened by PCR to find clones where cbh1 gene had been replaced by the construct. The primers used for the screening are shown in Table 2. The primers set forth as SEQ ID NO: 21 and as SEQ ID NO: 22 were used to verify that correct integration had taken place at the 5′ end of the cbh1 gene. Amplification of a 3592 bp fragment indicated successful integration to cbh1 locus at the 5′ end of the gene. The primers set forth as SEQ ID NO: 23 and as SEQ ID NO: 24 were used to verify correct integration at the 3′ end of the cbh1 gene. Amplification of a 1477 bp fragment indicated successful integration to cbh1 locus at the 3′ end of the gene. Primers SEQ ID NO: 25 and SEQ ID NO: 26 were used to verify complete deletion of the cbh1 gene by demonstrating no amplification of a 500 bp fragment from the cbh1 open reading frame.

TABLE 2
Oligonucleotide primers used for showing
correct integration and loss of cbh1 gene

| SEQ ID NO: | Sequence |
|---|---|
| 21 | ATCAGACCACGACGGGAC |
| 22 | CAGGTAGGAGGAGACCGACTG |
| 23 | TAGCGCGAATACTGCTGTGG |
| 24 | CATGCCGGAGTTGCTGAAG |
| 25 | GCTGACGCGAATGACACAG |
| 26 | CATGCCCTTGCCGTAGAAG |

The constructed C1 strains were grown in 24-well plates in the liquid medium as in Example 1. Mycelia were removed by centrifugation of 250 μl of the sample through a 0.65 μm MultiScreen filter plate (Merck Millipore) at 3500 RPM for 10 minutes. Production of Nivolumab by the transformants was confirmed by Western blot analysis. Transformants shown to produce Nivolumab were purified through single colony plating on selection medium plates as follows. Mycelium from a streak was suspended to 800 μl of 0.9% NaCl-0.025% Tween20 solution. Single colonies were obtained by preparing different dilutions (10⁻¹, 10⁻² and 10⁻³) of the suspension to 0.9% NaCl-0.025% Tween20 solution and plating 100 μl of the dilutions on selective medium. Production of Nivolumab by the purified transformants was verified by 24-well plate cultivation done as in Example 1 followed by Western blot analysis.

The purified transformant of the stt3 deletion strain M3210, positive for integration of the expression construct into cbh1 locus and producing both Nivolumab heavy and light chains, was stored at −80° C. and given the strain number M3480. The purified transformant of the cwh8 deletion strain M3211, positive for integration of the expression construct into cbh1 locus and producing both heavy and light chain of Nivolumab, was stored at −80° C. and given the strain number M3481.

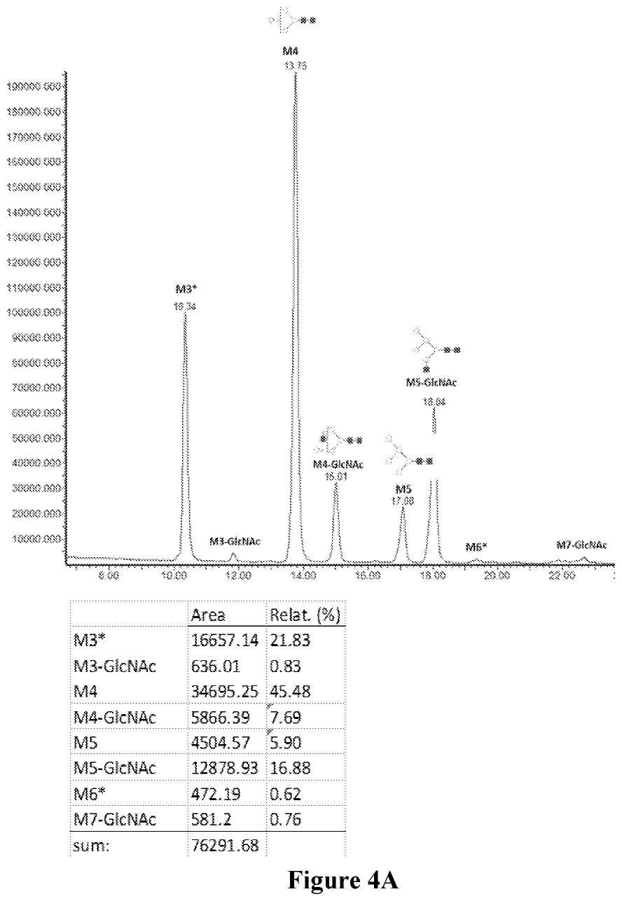

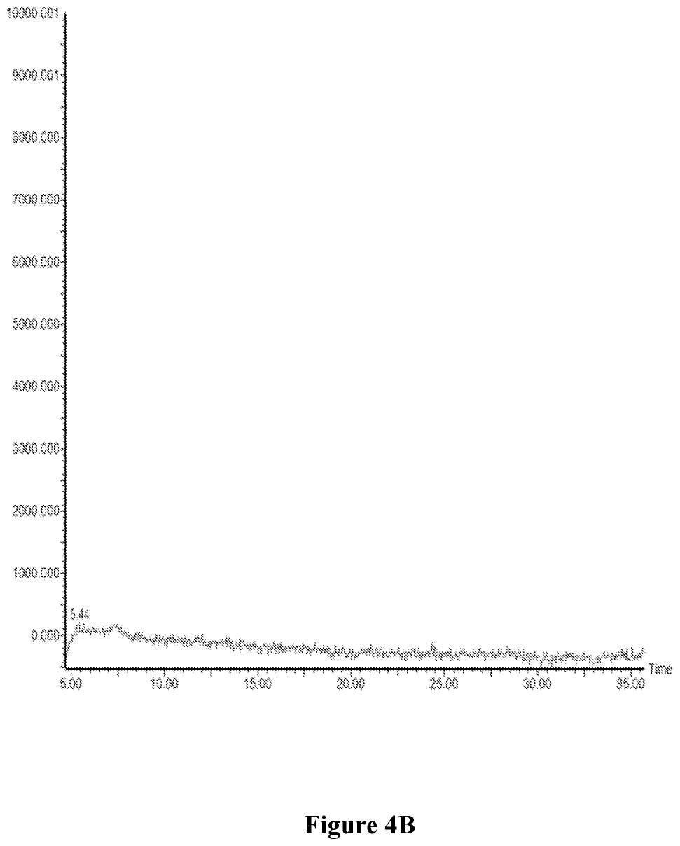

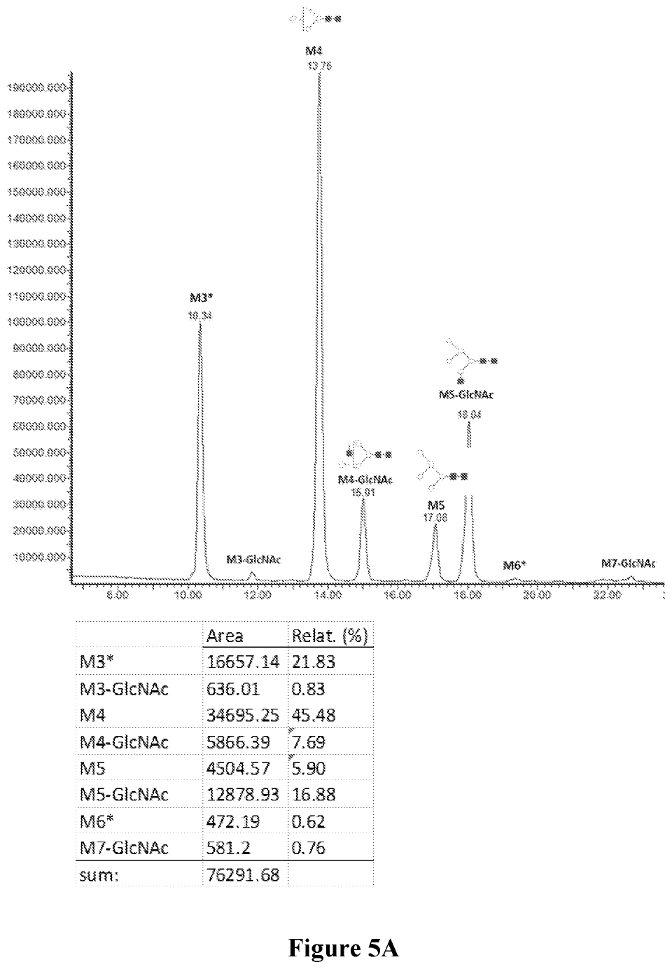

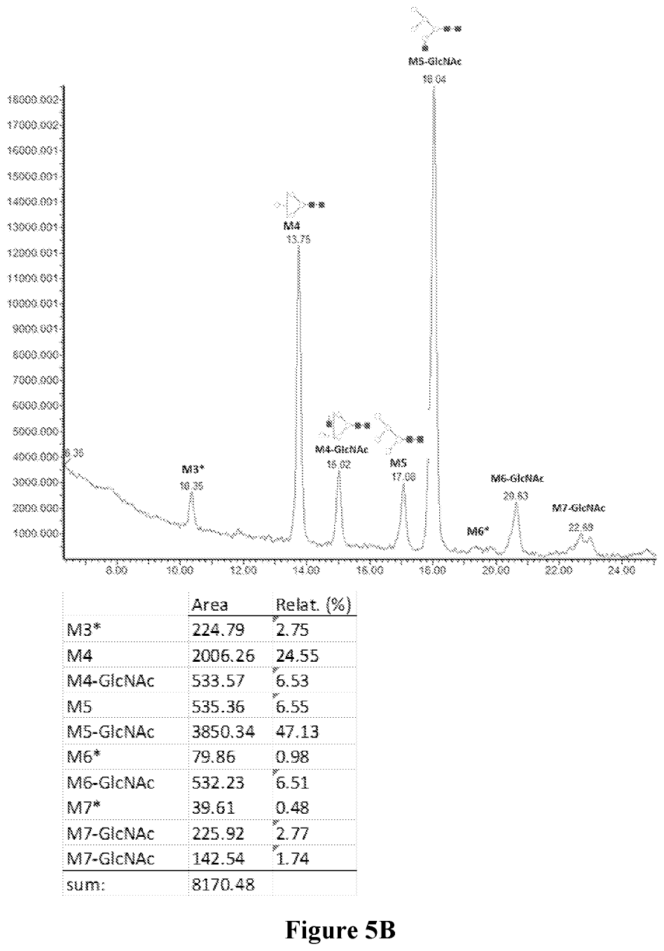

Strains M3480 and M3481 were grown in 1 L bioreactors in a fed-batch process in a medium containing yeast extract and glucose as the carbon source for 7 days. Nivolumab produced by the strains was purified through a 1 ml MabSelect SuRe protein A column (GE Healthcare) with ÄKTA Start protein purification system (GE Healthcare) according to manufacturer's protocols. Glycan analysis from purified antibody was done with the GlycoWorks™ RapiFluor-MS™ N-Glycan Kit (Waters) according to manufacturer's protocols. Nivolumab purified from the fermentation supernatant of a non-glyco modified strain M3242 was used as a control in the glycan analysis. No N-glycans could be detected from the Nivolumab purified from the M3480 culture supernatant whereas Nivolumab purified from the control strain showed a normal C1 glycan pattern (FIGS. 4A and 4B). Amount of N-glycans detected from the Nivolumab purified from the cwh8 deletion strain M3481 culture supernatant was approximately 11% of the N-glycan amount of the Nivolumab purified from the control strain (FIGS. 5A and 5B).

Peptide mapping for the purified Nivolumab was done according to the following method. 100 μg of samples were first buffer exchanged three times with 50 mM ammonium bicarbonate by Vivaspin 500 (10,000 MWCO, PES, Sartorius). Rapigest SF (Waters) was added to final concentration of 0.1% and dithiothreitol (DTT) to final concentration of 5 mM. Samples were incubated at 60° C. for 40 min. Next, iodoacetamide (IAM) was added to final concentration of 15 mM and the samples were incubated at room temperature for 40 min in dark. Trypsin (Promega) solution was added to each sample to a final protease: protein ratio of 1:50 (w/w) or 1 μl Trypsin (1 μg/ml) for 50 μg of protein. Samples were incubated at 37° C. overnight. Reaction was stopped by adding 4 μl 20% TFA after which samples were incubated for 30 min at 37° C. and centrifuged 5 min 10000 rpm. Finally, samples were evaporated by SpeedVac and reconstituted in 50 μl Water/AcCN/TFA 20/80/0.1 (v/v/v). Following LC-MS conditions were used. Instrument: Acquity UHPLC system, Waters (Milford, MA, USA) and Waters Synapt G2-S MS system (Milford, MA, USA). Column: ACQUITY UPLC Glycoprotein Amide 300A, 1.7 um, 2.1×150 mm (Waters) at 60° C. Solvents: A 0.1% TFA in Water and B 0.1% TFA in Acetonitrile. Gradient program: 0 min 10% A, 70 min 50% A, flow rate 0.2 ml/min. Mass spectrometry parameters used were positive polarity using the cone voltage 25 V and the capillary voltage of 3 kV, desolvation temperature 350° C., and source temperature 120° C. The data was collected at m/z range 50-2000 in MS^{E} mode using energy ramp 25-40 V. Data was processed using UNIFI software (Waters).

Nivolumab purified from the fermentation supernatant of a non-glycomodified strain M3242 was used as a control in the peptide mapping analysis. According to peptide mapping results, there are no N-glycans attached to the heavy chain of Nivolumab produced in the stt3 deletion strain M3480 (Table 3). Only 26.6% of N-glycosylation was detected from the heavy chain of Nivolumab produced in the cwh8 deletion strain M3481 (Table 4) whereas 95.8% of N-glycosylation was detected from the heavy chain of Nivolumab produced in a non-glycomodified C1 strain M3242 (Table 5).

TABLE 3
Peptide mapping results of the peptide harbouring the N-glycosylation
site of Nivolumab produced in the stt3 deletion strain M3480

| | | | | | | | | | Relative |
|---|---|---|---|---|---|---|---|---|---|
| | Possible | | | Charge | Obser. | | | | Intensity, |
| Chain | Assignment | Peptide | m/z | state | MW, Da | RT | Glycosylation | Place | % |
| HC | 286-294 | EEQFNSTYR | 1173.5188 | 2 | 1173.5188 | 16.07 | no Glyc | | 100.0 |
| HC | 286-294 | EEQFNSTYR | 1033.4218 | 2 | 2065.8363 | 36.64/37 | Man3 | N290 | 0.0 |
| HC | 286-294 | EEQFNSTYR | 1134.962 | 2 | 2268.9167 | 38.41 | Hybrid Man3 | N290 | 0.0 |
| HC | 286-294 | EEQFNSTYR | 1114.4511 | 2 | 2227.895 | 40.34 | Man4 | N290 | 0.0 |
| HC | 286-294 | EEQFNSTYR | 1215.9901 | 2 | 2430.9729 | 41.32 | Hybrid Man4 | N290 | 0.0 |
| HC | 286-294 | EEQFNSTYR | 1195.4763 | 2 | 2389.9452 | 43.2 | Man5 | N290 | 0.0 |
| HC | 286-294 | EEQFNSTYR | 1297.0157 | 2 | 2593.0242 | 43.96 | Hybrid Man5 | N290 | 0.0 |
| HC | 286-294 | EEQFNSTYR | 1276.5036 | 2 | 2551.9999 | 45.17 | Man6 | N290 | 0.0 |
| HC | 286-294 | EEQFNSTYR | 1357.5309 | 2 | 2714.0545 | 47.15/47.47 | Man7 | N290 | 0.0 |

TABLE 4
Peptide mapping results of the peptide harbouring the N-glycosylation
site of Nivolumab produced in the cwh8 deletion strain M3481

| | | | | | | | | | Relative |
|---|---|---|---|---|---|---|---|---|---|
| | Possible | | | Charge | Obser. | | | | Intensity, |
| Chain | Assignment | Peptide | m/z | state | MW, Da | RT | Glycosylation | Place | % |
| HC | 286-294 | EEQFNSTYR | 587.256 | 2 | 1173.5047 | 15.96 | no Glyc | | 73.4 |
| HC | 286-294 | EEQFNSTYR | 1033.4119 | 2 | 1155.4917 | 32.19 | Man3 | N290 | 0.9 |
| HC | 286-294 | EEQFNSTYR | 1114.4378 | 2 | 2065.8166 | 35 | Man4 | N290 | 6.4 |
| HC | 286-294 | EEQFNSTYR | 1215.9771 | 2 | 2227.8682 | 35.82 | Hyb Man4 | N290 | 1.5 |
| HC | 286-294 | EEQFNSTYR | 1195.4644 | 2 | 2430.9469 | 37.39 | Man5 | N290 | 1.6 |
| HC | 286-294 | EEQFNSTYR | 1297.0039 | 2 | 2389.9214 | 38.02 | Hyb Man5 | N290 | 12.4 |
| HC | 286-294 | EEQFNSTYR | 1276.4888 | 2 | 2593.0005 | 39.05 | Man6 | N290 | 0.2 |
| HC | 286-294 | EEQFNSTYR | 1378.0304 | 2 | 2551.9703 | 39.87 | Hyb Man6 | N290 | 1.8 |
| HC | 286-294 | EEQFNSTYR | 1459.057 | 2 | 2755.0535 | 41.34 | Hyb Man7 | N290 | 1.4 |
| HC | 286-294 | EEQFNSTYR | 1540.0824 | 2 | 2917.1067 | 42.79 | Hyb Man8 | N290 | 0.3 |

TABLE 5
Peptide mapping results of the peptide harbouring the N-glycosylation
site of Nivolumab produced in the strain M3242

| | | | | | | | | | Relative |
|---|---|---|---|---|---|---|---|---|---|
| | Possible | | | Charge | Obser. | | | | Intensity, |
| Chain | Assignment | Peptide | m/z | state | MW, Da | RT | Glycosylation | Place | % |
| HC | 286-294 | EEQFNSTYR | 1173.519 | 2 | 1173.5188 | 18.12 | no Glyc | | 4.2 |
| HC | 286-294 | EEQFNSTYR | 1033.422 | 2 | 2065.8363 | 36.64/37 | Man3 | N290 | 29.8 |
| HC | 286-294 | EEQFNSTYR | 1134.962 | 2 | 2268.9167 | 38.41 | Hybrid Man3 | N290 | 0.3 |
| HC | 286-294 | EEQFNSTYR | 1114.451 | 2 | 2227.895 | 40.34 | Man4 | N290 | 40.3 |
| HC | 286-294 | EEQFNSTYR | 1215.99 | 2 | 2430.9729 | 41.32 | Hybrid Man4 | N290 | 5.9 |
| HC | 286-294 | EEQFNSTYR | 1195.476 | 2 | 2389.9452 | 43.2 | Man5 | N290 | 4.5 |
| HC | 286-294 | EEQFNSTYR | 1297.016 | 2 | 2593.0242 | 43.96 | Hybrid Man5 | N290 | 14.4 |
| HC | 286-294 | EEQFNSTYR | 1276.504 | 2 | 2551.9999 | 45.17 | Man6 | N290 | 0.4 |
| HC | 286-294 | EEQFNSTYR | 1357.531 | 2 | 2714.0545 | 47.15/47.47 | Man7 | N290 | 0.3 |

The foregoing description of the specific embodiments will so fully reveal the general nature of the invention that others can, by applying current knowledge, readily modify and/or adapt for various applications such specific embodiments without undue experimentation and without departing from the generic concept, and therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed chemical structures and functions may take a variety of alternative forms without departing from the invention.

1. A genetically modified ascomycetous filamentous fungus capable of producing a protein of interest with reduced or no N-linked glycosylation, the genetically modified filamentous fungus comprising at least one cell having reduced expression and/or activity of STT3 and/or CWH8.

2. The genetically modified filamentous fungus of claim 1, wherein the at least one cell comprises at least one exogenous polynucleotide encoding the protein of interest.

3. The genetically modified filamentous fungus of claim 1, wherein the at least one cell has a reduced expression and/or activity of STT3.

4. The genetically modified filamentous fungus of claim 3, wherein the STT3 comprises an amino acid sequence having at least 75% identity to the amino acid of Thermothelomyces heterothallica STT3.

5. The genetically modified filamentous fungus of claim 4, wherein the Thermothelomyces heterothallica STT3 comprises the amino acid of SEQ ID NO: 27.

6. The genetically modified filamentous fungus of claim 1, wherein the at least one cell has a reduced expression and/or activity of CWH8.

7. The genetically modified filamentous fungus of claim 6, wherein the CWH8 comprises an amino acid sequence having at least 75% identity to the amino acid of Thermothelomyces heterothallica CWH8.

8. The genetically modified filamentous fungus of claim 7, wherein the Thermothelomyces heterothallica CWH8 comprises the amino acid of SEQ ID NO: 28.

9. The genetically modified filamentous fungus of claim 1, comprising at least one cell having reduced expression and/or activity of STT3 and CWH8.

10. The genetically modified filamentous fungus of claim 1, wherein the genetic modification comprises deletion or disruption of the stt3 gene such that the modified filamentous fungus fails to produce a catalytic subunit of the oligosaccharyltransferase (OST) complex.

11. The genetically modified filamentous fungus of claim 1, wherein the genetic modification comprises deletion or disruption of the cwh8 gene such that the modified filamentous fungus fails to produce a functional dolichyl pyrophosphate phosphatase.

12. The genetically modified filamentous fungus of claim 1, wherein the ascomycetous filamentous fungus is of a genus within the group Pezizomycotina.

13. (canceled)

14. The genetically modified filamentous fungus of claim 12, wherein the ascomycetous filamentous fungus is of the species Thermothelomyces heterothallica (also denoted Myceliophthora thermophila).

15-16. (canceled)

17. The genetically modified filamentous fungus of claim 1, wherein the protein of interest is selected from the group consisting of an antigen, therapeutic protein, antibody, enzyme, vaccine and structural protein.

18. The genetically modified filamentous fungus of claim 1, wherein the protein of interest is a secreted protein.

19. The genetically modified filamentous fungus of claim 1, wherein the ascomycetous filamentous fungus is a strain further modified to delete one or more genes encoding an endogenous protease.

20. (canceled)

21. A method for generating an ascomycetous filamentous fungus that is capable of producing proteins with reduced or no N-glycans, comprising:
a) reducing the expression and/or activity of STT3 protein of the ascomycetous filamentous fungus; and/or
b) reducing the expression and/or activity of CWH8 protein of the ascomycetous filamentous fungus.

22. The method of claim 21, comprising:
c) deleting or disrupting the stt3 gene of the ascomycetous filamentous fungus as to reduce the production of a functional catalytic subunit of the oligosaccharyltransferase (OST) complex; and/or
d) deleting or disrupting the cwh8 gene of the ascomycetous filamentous fungus as to reduce the production of a functional dolichyl pyrophosphate phosphatase.

23. (canceled)

24. A method for producing a heterologous protein having reduced or no N-glycans, the method comprising:
a) providing an ascomycetous filamentous fungus genetically modified according to claim 1, wherein said fungus comprising an exogenous polynucleotide encoding a heterologous protein of interest;
b) culturing the ascomycetous filamentous fungus under conditions suitable for expressing the heterologous protein; and
c) recovering the heterologous protein.

25. (canceled)

26. A recombinant protein produced by the ascomycetous filamentous fungus genetically modified according to claim 1.

27. (canceled)


SEQUENCE LISTING (29 sequences)

SEQ ID NO: 1 - DNA, 8247 bp, Artificial sequence
  Synthetic sequence
aggcgttttcgtgggtaatgggatggttgtatctaggggcactcctggcgcggtttataaaaacatttggcactcaacacattgcatcatcccgggaattctgcttcagggtttggggtgttatacgaggtcaatgatttctcggtcccggcttggtatctataccactgccgaatgaaagcgctgcgctgaacacccagcctcagaccaggccctcgttggtgatctacatccggtgatgattgctgggattgtgtgccggcaattgggcctcgaccgcgtccagtgatggatcagtgaacccagtcgcgagcgggttcgtagttcaccttataccccgcctgagatatgatacagttacccaaagccagggcgttctaggtttgctccatccgcgatctgatgtgatagatccgtgccccacacaatcgccaagagtcccatcagatcaatggatctctgttttcccaaggttatgcacatatatatggtcatggacctggtgatagatccatcaacatgagttgtttcgcagttctccgggagttacgcctcgcggatcttcttaatcccagatctctccctctgtatctttcggcctctgcattgccacccctatacgaagtattggaccctttgtactgcgtacggatttggttttctgctctttctttggcccctccaactttctacatattaatcggacttctgccgggcggtgaagcgttgagcaacgaggctgagtgggtcttgggcgcgaatcaagcatgccaaggcggacccctggagcattccactccagttgcatgcagaagctccaaactgtcctctccaaaggccagagctcgctcgtctgcgcttgtccttggtgttagcctccgacttcaactgggcgagaggcagcaccacaccccgcaaacgcaggccaccggcgcgccgttggcggtaacagcaggtgccgttcgctgctgctcggtccttttcccgaaagtaccctgccttcctggcctcgacctccgcctgcggggctctgggcactgtcaaactttcgcagttttgcacccgcagcgattcgagcgggacccgcttcgccaacgcggatcaggagaggtaaaagtctcccctgtcctttctgccggagcatcctccgtccgtcgttcgctggttgttgtgtgtgtgtgtgtgtacagtactgtaggtacatagtgctcaccccagggcatttccccaatccgcgattgcgcataccataggcgtcatctgaatctgcgttacgtccggataacactacgcagtacgaaggagtcggtgctactacgcccgcaagttcaagtctacggtgtggctaaggtgctcaagggcatagttacacgcaccccgggcgagcttctcgcacctcgaccctttgccggggcgtcactggtccgacaactcataatccaccatcgcttgggcaggcctgcatcccccgtgggtcgcatccaagccgccggtaaataccggccgacacacacacacacacacgcaccctgtctagcgcgacgctacaaaagagaccggcaggcactccccgcgaccgcttgttctggccatcccgtctctcgcctctgcagttatcgcctgcctcatctctgcgtcctgataacttttgtcacctctgatccccccccgacgagacggtctcgccgaccaggacccgagatggcggaccgccatcctactctgtcccagtccttcgccgagcgggcgaagacggctagccacccgctcacccgctacctctttcggctcatggacctcaaggcctcgaacctgtgcctgagcgccgacgtgtccaccgcgcgcgagcttctgacgctggccgaccgggtcggcccctcgatcgtcgtgctcaagacgcactacgacctgatctcgggctgggactacaacccgcaaaccggcaccggcgcgaagctggccgccctggcgaggaagcatggcttcctcatctttgaggaccgcaagtttgtcgacattggtaagacggtgcagatgcagtacacggctggcactgcgcgcataatagagtgggcgcacatcaccaacgccaacatcgacgccggcaaggacatggtgcgcgccatggccgaggcggccgccaagtggaaggaacgcatcaactacgaggtcaagacctccgtcacggtgggcacgcccgtctcggaccagttcgacgatgcggaagagcaagcgcagtggccgcagcaccagcagcaccagcaccagcaccagcaccagcaacagcgagatgaaaaaggtgggccccgcaggctcggcactcgggaggagcagcaccaacaggacaacggagacggtgacggccggaaagggagcattgtctcgatcactacggtgacgcagtcatttgagcccgctcactccccacgcctgtccaagagcaacgagctgggcgacgacgccgtcttccccggcatcgaggaggcccccgtcgaccgcggcctgcttctgctcgcccagatgtcgtccaagggctgcctcatgaccaaggagtacacccaggcctgcgtcgaggccgcgcgcgagcataaggattttgtcatgggcttcgtctcacaggagtcgctcaactcggccccggacgacactttcatccacatgacccccggatgcaagcttccgccgccaggcgaggacgaagagagcggccagatcgagtttaaacggcgcgccgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacggtttaaac

SEQ ID NO: 2 - DNA, 8245 bp, Artificial sequence
  Synthetic sequence
gaacctgtgcctgagcgccgacgtgtccaccgcgcgcgagcttctgacgctggccgaccgggtcggcccctcgatcgtcgtgctcaagacgcactacgacctgatctcgggctgggactacaacccgcaaaccggcaccggcgcgaagctggccgccctggcgaggaagcatggcttcctcatctttgaggaccgcaagtttgtcgacattggtaagacggtgcagatgcagtacacggctggcactgcgcgcataatagagtgggcgcacatcaccaacgccaacatcgacgccggcaaggacatggtgcgcgccatggccgaggcggccgccaagtggaaggaacgcatcaactacgaggtcaagacctccgtcacggtgggcacgcccgtctcggaccagttcgacgatgcggaagagcaagcgcagtggccgcagcaccagcagcaccagcaccagcaccagcaccagcaacagcgagatgaaaaaggtgggccccgcaggctcggcactcgggaggagcagcaccaacaggacaacggagacggtgacggccggaaagggagcattgtctcgatcactacggtgacgcagtcatttgagcccgctcactccccacgcctgtccaagagcaacgagctgggcgacgacgccgtcttccccggcatcgaggaggcccccgtcgaccgcggcctgcttctgctcgcccagatgtcgtccaagggctgcctcatgaccaaggagtacacccaggcctgcgtcgaggccgcgcgcgagcataaggattttgtcatgggcttcgtctcacaggagtcgctcaactcggccccggacgacactttcatccacatgacccccggatgcaagcttccgccgccaggcgaggacgaagagagcggccagatcgagggcgacggcctcggccagcagtacaactcgcccagcaagttgatcaacatttgcggcaccgacattgtcatcgtagggcgtggcatcaccgccgccggcgacccgccctccgaggctgagaggtacaggagaaaagcctggaaggcctatctggcgcgtctggcgtgatttggggggagggggagaggagatgggggacgggaggggtcgccttggtcagtcttgtgcgtgtcctgcagcggattcgtcaccggggcagcacccaaaagagggagaaaaaggggaaaaaaaataaataaataaaaagggttaagttgttgaaaaaagtgttgtgagctctctggcaaggcgcgccccccacacaatcgccaagagtcccatcagatcaatggatctctgttttcccaaggttatgcacatatatatggtcatggacctggtgatagatccatcaacatgagttgtttcgcagttctccgggagttacgcctcgcggatcttcttaatcccagatctctccctctgtatctttcggcctctgcattgccacccctatacgaagtattggaccctttgtactgcgtacggatttggttttctgctctttctttggcccctccaactttctacatattaatcggacttctgccgggcggtgaagcgttgagcaacgaggctgagtgggtcttgggcgcgaatcaagcatgccaaggcggacccctggagcattccactccagttgcatgcagaagctccaaactgtcctctccaaaggccagagctcgctcgtctgcgcttgtccttggtgttagcctccgacttcaactgggcgagaggcagcaccacaccccgcaaacgcaggccaccatttaaataaaggtcagcagcgggtggatttgtgtacaaagaagtatgttagagttgattcttttattttgcgacatgcggattgggaaaagggcatcgggtcggtcgtattgtgtacaacacaagcacgttgattgcgctacatggtgccacagaacacatggttgaaagtgacctcgtccaacggcggctccaactgtgagcaaccagccaaatgtgcattcatcgtcgcgcccgaacagagatcgaagtgtcgaagaagaagcggcagcgatttgcgcgatttccagccatggtaagtaaacaaggaaaaaaaaaaaaaggggggggggacagcaggcgttgctgttaaagtgtcaagaagcgctcaatgatttctgcctctctcatgagtcttcctcgccctatgatttctgcctctctcatgagtcttcctcgccctttgacagctcccaccactgcgggttctttaccagcctttcaactgatctctttcatcccattagcatgcaacaggtcccgtgggcctaggcgcccgggcgaaatttcagtgcagcatttgagatgagcccagttcgcacaactttgcccatacgggaacattccctgcttgttgcgtttgttcttatagcctacctatcagccattccatcctctcacagctgttcccgctgctcaacttcgtttccttcctcctctccctacataacctgccgattctgttcggacctacggattcctacaatagtgtagtcaacgaactctggctaccggaccgcaagatgcatttcagtgtgtaagtcagtctcatgggtttgctggtttcctttcccttcgagacaggtcgctagtttcttccgttcgctagctttctgccaggaggccaggagggctcccaagacgttccgccatggctacattgctcctcgctgactgttgtttaaacgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgggcgcgccgtttaaac

SEQ ID NO: 3 - DNA, 8317 bp, Artificial sequence
  Synthetic sequence
atagcgcgatctgtgcctccttgtatgtatgtatgtagtccgtacaacatacagtactatacgggacacgcattccatagagcggattacactagaactgcccattctgcgaacctgagaaccaaggtcacataccacgttcttgatttgagatttccacaaatacatcgttcgcacgttggccttctgtctgacttagaacgccaagtaaccgtctctaaaagcgcttagggcctagaagctgcgcaagtaccctacaagtatgctttgtatgtacatacatacatacatacctacgtccatcaggtatgtacctaagtcaggtcgcttgagggccttccatacttgggagaagcggatgcaattacccacatgataatagaggcacctacagaccgcccacaattgtgtagttttggcaagcttcgtcactcagcactcctttattctcttctttaatgaattatatgaagaactatcagtcaagtaacccagtgcattgtgtgagatcaatacgatatgcagtgaaaggtgcatttaccgacttagcgcatcacgcgggaatgggtggagtactacggataccgctacgagttggaggtacagtagttaccccggggggaaggggttggcggtttgggggtttggcctctgccgattggccaacttctgccaaggccggcctttctgccgttcacatttcaacggcgaagggcagctgcttgaaacaaacttcggcccctaacgacggcgcacgcaaactcgcggtaaccacttacccggccaggccaactgatatgagggcttgaaagagccaaaccaacgatgcagcgaactaacaagcagcgctgctgataaaacgatcagaaagagcctcgacctctctcctttttttctagagccgtgcgcatacttcttgctgctcgcaaccaacaacaacagtacagaacacaacaaaatagcagagttccgagaaacagtgcagaagagagttgccaccggcgcgccgttggcggtaacagcaggtgccgttcgctgctgctcggtccttttcccgaaagtaccctgccttcctggcctcgacctccgcctgcggggctctgggcactgtcaaactttcgcagttttgcacccgcagcgattcgagcgggacccgcttcgccaacgcggatcaggagaggtaaaagtctcccctgtcctttctgccggagcatcctccgtccgtcgttcgctggttgttgtgtgtgtgtgtgtgtacagtactgtaggtacatagtgctcaccccagggcatttccccaatccgcgattgcgcataccataggcgtcatctgaatctgcgttacgtccggataacactacgcagtacgaaggagtcggtgctactacgcccgcaagttcaagtctacggtgtggctaaggtgctcaagggcatagttacacgcaccccgggcgagcttctcgcacctcgaccctttgccggggcgtcactggtccgacaactcataatccaccatcgcttgggcaggcctgcatcccccgtgggtcgcatccaagccgccggtaaataccggccgacacacacacacacacacgcaccctgtctagcgcgacgctacaaaagagaccggcaggcactccccgcgaccgcttgttctggccatcccgtctctcgcctctgcagttatcgcctgcctcatctctgcgtcctgataacttttgtcacctctgatccccccccgacgagacggtctcgccgaccaggacccgagatggcggaccgccatcctactctgtcccagtccttcgccgagcgggcgaagacggctagccacccgctcacccgctacctctttcggctcatggacctcaaggcctcgaacctgtgcctgagcgccgacgtgtccaccgcgcgcgagcttctgacgctggccgaccgggtcggcccctcgatcgtcgtgctcaagacgcactacgacctgatctcgggctgggactacaacccgcaaaccggcaccggcgcgaagctggccgccctggcgaggaagcatggcttcctcatctttgaggaccgcaagtttgtcgacattggtaagacggtgcagatgcagtacacggctggcactgcgcgcataatagagtgggcgcacatcaccaacgccaacatcgacgccggcaaggacatggtgcgcgccatggccgaggcggccgccaagtggaaggaacgcatcaactacgaggtcaagacctccgtcacggtgggcacgcccgtctcggaccagttcgacgatgcggaagagcaagcgcagtggccgcagcaccagcagcaccagcaccagcaccagcaccagcaacagcgagatgaaaaaggtgggccccgcaggctcggcactcgggaggagcagcaccaacaggacaacggagacggtgacggccggaaagggagcattgtctcgatcactacggtgacgcagtcatttgagcccgctcactccccacgcctgtccaagagcaacgagctgggcgacgacgccgtcttccccggcatcgaggaggcccccgtcgaccgcggcctgcttctgctcgcccagatgtcgtccaagggctgcctcatgaccaaggagtacacccaggcctgcgtcgaggccgcgcgcgagcataaggattttgtcatgggcttcgtctcacaggagtcgctcaactcggccccggacgacactttcatccacatgacccccggatgcaagcttccgccgccaggcgaggacgaagagagcggccagatcgagtttaaacggcgcgccgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacggtttaaac

SEQ ID NO: 4 - DNA, 8503 bp, Artificial sequence
  Synthetic sequence
gaacctgtgcctgagcgccgacgtgtccaccgcgcgcgagcttctgacgctggccgaccgggtcggcccctcgatcgtcgtgctcaagacgcactacgacctgatctcgggctgggactacaacccgcaaaccggcaccggcgcgaagctggccgccctggcgaggaagcatggcttcctcatctttgaggaccgcaagtttgtcgacattggtaagacggtgcagatgcagtacacggctggcactgcgcgcataatagagtgggcgcacatcaccaacgccaacatcgacgccggcaaggacatggtgcgcgccatggccgaggcggccgccaagtggaaggaacgcatcaactacgaggtcaagacctccgtcacggtgggcacgcccgtctcggaccagttcgacgatgcggaagagcaagcgcagtggccgcagcaccagcagcaccagcaccagcaccagcaccagcaacagcgagatgaaaaaggtgggccccgcaggctcggcactcgggaggagcagcaccaacaggacaacggagacggtgacggccggaaagggagcattgtctcgatcactacggtgacgcagtcatttgagcccgctcactccccacgcctgtccaagagcaacgagctgggcgacgacgccgtcttccccggcatcgaggaggcccccgtcgaccgcggcctgcttctgctcgcccagatgtcgtccaagggctgcctcatgaccaaggagtacacccaggcctgcgtcgaggccgcgcgcgagcataaggattttgtcatgggcttcgtctcacaggagtcgctcaactcggccccggacgacactttcatccacatgacccccggatgcaagcttccgccgccaggcgaggacgaagagagcggccagatcgagggcgacggcctcggccagcagtacaactcgcccagcaagttgatcaacatttgcggcaccgacattgtcatcgtagggcgtggcatcaccgccgccggcgacccgccctccgaggctgagaggtacaggagaaaagcctggaaggcctatctggcgcgtctggcgtgatttggggggagggggagaggagatgggggacgggaggggtcgccttggtcagtcttgtgcgtgtcctgcagcggattcgtcaccggggcagcacccaaaagagggagaaaaaggggaaaaaaaataaataaataaaaagggttaagttgttgaaaaaagtgttgtgagctctctggcaaggcgcgcctgtgtgagatcaatacgatatgcagtgaaaggtgcatttaccgacttagcgcatcacgcgggaatgggtggagtactacggataccgctacgagttggaggtacagtagttaccccggggggaaggggttggcggtttgggggtttggcctctgccgattggccaacttctgccaaggccggcctttctgccgttcacatttcaacggcgaagggcagctgcttgaaacaaacttcggcccctaacgacggcgcacgcaaactcgcggtaaccacttacccggccaggccaactgatatgagggcttgaaagagccaaaccaacgatgcagcgaactaacaagcagcgctgctgataaaacgatcagaaagagcctcgacctctctcctttttttctagagccgtgcgcatacttcttgctgctcgcaaccaacaacaacagtacagaacacaacaaaatagcagagttccgagaaacagtgcagaagagagttgccaccatttaaatggatcccggttagagcaggtgggaaaccatttgtgaatgggttggcatgagggctcaaatgcaagtaatctatttactctctctaatcgatgcaaatagtttcgaccgttcctgataaagcacattacaactcagctctactttttatcaacatttgaagcactgactcgtccaaataaaccgctgctgtctcatcggcttgccccggaaccacaagtagcacggtttcgacatcttaccggtgatacacaccgggattgtcataccgatacccggcactaccattcgggtaactcccgagcggaatgtcattgccggcgccatacggtgacttggtcgagccgttgcccaacccgccatacccgccgccgctgcccacccgggtctgctcggtcgcatagctattgcgcagatcctccggggtagagtgctggggcagcgcgttgggatccggaatgccgtccaccggtgcagtgcgcttccggccgaggccgaaccagttaaagcgcttgcggcggccgtagccctcggtgtagttgttggccggggacgggccgaagcgcgcctccttacggcggtggcgaacgagggccaggctaacgaacggagtgatggcgaacaagatgctatcgtcaactcacatgtcagcctacgagaggtattctcgtcgagggaagggaaagagggccggggggagggggaagggaggaaatgaggacgtacactgcaacgatcgatactgccaaacatgccgactccatcttgcaagccgtcctcaaactaggcagcgccgtaaacccgccgctgccgttatccaccacattggtattcgcatctcctttgccatacaccgtgtcgacctccccgttacaagacgacgccccgccgcggttcgcagtagcaatgtaaatgaagccgccgatgaaggccacatcgaggaccatcgtgatgaatgacgggaatgagcggccgggtacgcagcagagcatgagcagggcaaggatagtgtagaggacaccaacgccagagatgcctgcgaccgcccgtgcccaagtagggatgtgcatattatggttggtaagagtcgcgagaaagtaggagaagagcgcgagcacgatcgcggcgcagcagaactgaatggctcgcaagaggaacatcaagccccgaagggcaatgcctttggccgcgcccatggcgttggtttaaacgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgggcgcgccgtttaaac

SEQ ID NO: 5 - DNA, 20 bp, Artificial sequence
  Synthetic sequence
ccgtcaaggtacgagaaacc

SEQ ID NO: 6 - DNA, 20 bp, Artificial sequence
  Synthetic sequence
agtttgacagtgcccagagc

SEQ ID NO: 7 - DNA, 20 bp, Artificial sequence
  Synthetic sequence
ttcttaacaccgagggttgg

SEQ ID NO: 8 - DNA, 20 bp, Artificial sequence
  Synthetic sequence
agcctggaaggcctatctgg

SEQ ID NO: 9 - DNA, 20 bp, Artificial sequence
  Synthetic sequence
acaacaacctcttccctcct

SEQ ID NO: 10 - DNA, 18 bp, Artificial sequence
  Synthetic sequence
tcgtgaaagcctcctcca

SEQ ID NO: 11 - DNA, 18 bp, Artificial sequence
  Synthetic sequence
acaacgaagccattgcca

SEQ ID NO: 12 - DNA, 20 bp, Artificial sequence
  Synthetic sequence
caaccatgtagccgtaggag

SEQ ID NO: 13 - DNA, 20 bp, Artificial sequence
  Synthetic sequence
gtttcggtttttgagaacgg

SEQ ID NO: 14 - DNA, 20 bp, Artificial sequence
  Synthetic sequence
cgtcctaaagcgtccaatcg

SEQ ID NO: 15 - DNA, 20 bp, Artificial sequence
  Synthetic sequence
ggtgacgtatctgatgaggg

SEQ ID NO: 16 - DNA, 20 bp, Artificial sequence
  Synthetic sequence
cacaaatcttcctcgaccac

SEQ ID NO: 17 - DNA, 20 bp, Artificial sequence
  Synthetic sequence
gtggtcgaggaagatttgtg

SEQ ID NO: 18 - DNA, 20 bp, Artificial sequence
  Synthetic sequence
tcacaaatggtttcccacct

SEQ ID NO: 19 - DNA, 15882 bp, Artificial sequence
  Synthetic sequence
tgcgcaagcgcccttcatctcttcagccgagaatctgttgcctaattacatagtagatgctgggaacagtctgagaagcctgcgcaggttcttcattaattaccaagaacgcgggagaggtgggaagacgccgaaaactcgacgcggaccattcgctgttttccgatcttacaaaaaaaaggtatccgatttggggaacgtcgatgaaagtattgcaaaagtgacgagagttgcgcaactaactcgctgccgaagaagctgcggaagaaagagaacaccgaaagtggaataacgttacggatgtcctgacctcaaagttgaaaccagcccttcctgctctatttgggaaagcggcttgcccttgaatgcgctgcactgtggcacgactaccagtgatcgggaggagcaaactaccctggtccgttccttggtggggcggcactaggcccaacttagggtgatcggaggtcgatgccgcggtcctcgttggtctgggctcttctcatttcccggtttgcaccccccgttgcacctgctgatcgcccgccaacgccgatgaggttgcgcccagaccgacaatcaccgcggctgcattcccaagtatattgaagatggcaccaggtacccggttttgcgtcccagtcgtttggtgccaaatttgggagtttttgagcctcaagatctggggaaatcgacctcaacttccatacaagttaaagtcgcacacacggcgagttccacgaagagacacatttttttctgaaggcctctctccccgcacatcagaaaccaccaaataccaagactgcagaagccggggtaagtgggccaccgggactacactaaaatgcggggagaagcgagatccgttgcgaagggaagggatggggtgtgctgcggctttctccgctctcgtgcgccttttgcttgaatctagtgtacaccagggtaggctccgaaggagtatctacggcagcgctgttcgtgctgcgttgagagtcagggcggagacgagcaggcgacaggagcctcgcaccggcacttcggatcgcatttgcgcggagcgtcaaatacgctcttctgcggtcatcagagagcatcgtgaaccaaggttcttccgcagggcggcctgggcttcgcagagtcgcactcggcggacgccttccgtgtcacccctgataacctggctgccgcgcccagactcctccaatgaggtgtgtggttgccctcgccgacccttcagcaaccttaatcgcttccatcgcacggctccacgtcctcgaacgatgccctcagtccgtgcccggccgtggcaaccataacgtgacatcgccgcccagcctactagccgctatcgaccggttaggcttgtcaccgcagcgcccattctccatcgggcctctactctgatccacctcacccaccgcaagcactagcgagcctcaccagagtgcaagcgacacgacccgcttggcccttcgtccttgactatctcccagacctcttgccatcttgccgacgccgcccccttttttttctcctccccctgccggcaggtcggtggccccagtcccgagatggcattgctccgttgtccatgacgacccatcattcgatggctgactggcacactcgtcttgtttgagcatcgacggcccgcggcccgtctcccacggtacggaacctcgttgtacagtacctctcgtaatgatacccaacaccggggccgagcgctgggagggcggcgttcccgagaagccgggaaggcggctggccggctgacctttgtgacttggcgatggatgcggccatggagaatgtccgtccgaagcgacgcgacaattagcctggctaccatcgatataaattgggtgattcccagctcttgatgggcgtgtcttctgcctggcagccctcgtcttcagatcaagcaactgtgtgctgatcctcttccgcccgccggcgggttgccacggtcacggaatgatggagcgggtctgacacaccaaacactcgataagcagagcggcgatgtccatgctccggaacagccagcccgagttgccgtcgcccatagtgttccaagtaagcgtatgggacgagaagaaagaagagaaaagaagaagagagccgcccggctcgttcagagtcggccgcacggttcggtttcctgggagcgaagggacgtgaatcgccagagcgaccgaggtttagccacggcaagaaagcaaaatccggagttttgccagagagggcgcgcatcgctaaagtgctccttggcccggatgcgtctcgtctcgcctcagagagatggataccccgtgccttggctagaaagcctggcttgcgagatgctcctctcggcgcgggcggcgtggtgccgtggttcgtttgctgagctagaacgacgcccctgcgcgtgaccagcgtgtttttggtttgttcacccccagaatcagccgaggtttacccagccggagcgctcgtcaggtccttcacgggccgtggatccggaaactatgcgaaggtggagagagcgactcggagcgactttcgcgattattctggcgagttttcgagggcgcggtaactgcgaaagctgaccttcttcaattcaatcaacaatggaaggagaggtcgaattcggaggtttaccccccccccgcgccaaaatagtctctcccaggtgcccatgtaaggcacgggaatacctgcctaacttggggttgggaaagaaaagaaaggcaggaaaaaaatacactatcataccatgctcaagctttctctttccagaaacatgtgtttcgggttctccttccgtgctcccagagctggccctttggcccgttgggggttggcggggcccaagggtgcgctagtggggctcctcagtctccgcaatatcgtgcatcgcaacgcgcaaggcccttgcccactatcaacagccccccggattgctgaccgttggccattcacggcccttcgtttgccagtccttcgccagggtcaacctaccccgcggtggggggttgttcttggatccttgtcgaggccccggctgcccacatcgcccacgctgtgcactcagcgtaacacagggcccggatctctcaagcgatgcccagctttttttcatcggtgttgacggtccgaaactcgcgggagagatgggggcagatcatggcgggaaacggccgtgatggttcctggatataaaggagatcaggccttccctcctcggctcattggggcctactagcacatcatcatccgtcttccatccctcctcagaacttccttccccttcctcctatccacctttcccttactcacacagacaatcgtccatcgtccaccatgtacgccaagttcgccaccctggccgccctggtcgccggcgccgccgccgagatcgtcctcacccagtcgcccgccaccctctccctgagccccggcgagcgcgccaccctgtcctgccgcgcctcccagtcggtctcctcctacctggcctggtaccagcagaagcccggccaggcccccaggctcctgatctacgacgcctcgaaccgcgccaccggcatccccgcccgcttcagcggctcgggctccggcaccgacttcaccctcaccatcagctccctggagcccgaggacttcgccgtctactactgccagcagtcgagcaactggccccgcaccttcggccagggcaccaaggtcgagatcaagcgcaccgtcgccgccccctcggtcttcatcttcccgccctcggacgagcagctcaagtcgggcaccgcctccgtcgtctgcctcctgaacaacttctacccgcgcgaggccaaggtccagtggaaggtcgacaacgccctccagagcggcaactcccaggagtcggtcaccgagcaggacagcaaggactccacctactcgctgtcctcgaccctcaccctgagcaaggccgactacgagaagcacaaggtctacgcctgcgaggtcacccaccagggcctgagctcccccgtcaccaagtccttcaaccgcggcgagtgctaaggcaggcaggagttggagtatgagggtagccgctgatggctattcttcccacgtttttgtgtgtttcctcttcatttttttttctcttgccgcaacatgacggctcctgtctctgaagggaacccctgaaattcagggttatcatgacttggttacgaatgagctacgacatgttcaattgagtgactctttactaccaaagtactgctaccatgacactcgaatcgtctcgtgactgaaaggagaatcatgttggcattggttcgcgtagtacggagtaacgacaacggcattggtcaacatctggcaggtatttgaggtagaatataccaacctgcctgaggctctcggtatcaagatttggaaggccaaagggttggatgagcacttgagagcaaagtcggactactggctgaagaaggtaaacaaactaacgtacagtacctacttaacttatgatacacgtccgcgcgcgggcgcgccttatcggacggcctgatttgcggcatgcctcatcgcggcgaaactgctgataagacctgctgccgcagtccgcccattcctgtgtctgacgcacaccgattggggcatgtccgcggaaaaagagacgatcctctccactaggtggtttccgcctttaacggatcgaggaaacattagttgttagtagtaacacgccttgaacgccttgatctccggggctcctcctcggacgaccgcggaccgccggtcagagtgggtgggtacaccacactacacgatactatcgcgcatcgtcgttgtccctgctctctccccatatggcgtcacggggtagtatatattctacacggagctggagccaaggcaggcccaccgtacggattacaccgccatggttccaagtttcttcgccattgaatctgttattcgtgtactagtagatcaagccattgccggttgccgatatcatgcaactccggtcaggccacggagaagccatgcccgctacgggttgatgttccctgttccctttcataaacgggaataagacgaagtgcgcggcgggagttgggagcccatacccgacctcgatgctttccccggttcttccattccctagtactagtgtagctatttcagagtcttcatgccagcccttccccggaggcccctggcgcccccccaccccttcctctcctggcgagagtgcacacagtctttttgtacagtcttggtcccatctagtgggcgttttccccttctttcgtccccattgtccgtagagagagtgttgggtccgaccgcctaccgacgacaatggagatggcgccctcgacgacctcctcctcttcttcccttgaagggtccccgatgaccttcccatcacacctgccgccaacccctccgctgcaggattttgacgaatatcagaaactaggtctcagcctgccgacagatggcctcggacgatccgtccctccgtaccctctccctccgccgtcgaggcagaacaaggtgctgcccgaggacctcaaaacgccggatgaccacgtggagcgggacccccgcctgatccggctcaccggagtccaccccttcaacgtcgagccccccctgaccgacctctttgacgaaggcttcctgaccaccaaggacctgcactacgtccgcaaccatggcgccgtcccccgggtcgaggactcatccgtgctcgactgggacttcaccgtcgagggcatggtcgcccacccgatcacgctcacgctgcgcgacctgatccggacgtacgaccaggtgacgtacccggtcaccctggtgtgcgcgggcaaccggcgcaaggagcagaacgtggtgcgcaagaccaagggtttctcgtggggtgccgccggcctgtcgacggcgctgtggacgggcgtcgccctgcccgacctcctcgcccgcgccgccccgctgcgcggtgcgcggtacgtctgcttcgagggcgccgaccgcctgcccaacggctgctacggcacctcggtccgcctcggctgggccacggacccggcgcgtggcatcatgctcgcccaccgcatgaacggcgagccgctgcccccggaccacggcaagcccctccgcgtcatcatcccgggccagatcggcggcaggagcgtcaagtggctcaagcgcatcatcgtcacggcgaagcccagcgacagctggtaccacatctacgacaaccgcgtgctgcccaccgtgatcagccccgaggagagcgccgaccggcccgacgtctggaaggacgagcggtacgccatctacgacctcaacaccaacagcgccatctgctatccggcgcacgacgagagggtgccgctcgggatcgcgcgcggggacgcggcgacgtacaagctgcgggggtacgcctacgccggcggcggcaagagggtgacccgtctggaggtgacgctcgaccagggccggacgtggatgctggcgaccatgtcgtacccggaagacctgtaccggctcgcgcccgagggggagacgctgtttggcgggaggctggacgtgtcctggcgcgagacgtgcttctgctggtgcttctggtcgctggacgtgccgctgtcgcggctgcgagacgcccgcgacgtcatgatccgggcgatggacgagtccatgatggtgcagccgcgggacatgtactggagcgtgctgggcatgatgaacaacccatggtaccgggtcgtcatccacaaggagagcgacacgttgcgattcgagcaccccacccagccggcgctgatgcccggcggttggatggagcgggtcaagaaagaagggggcaaccttaccaatgggttttggggcgagaaggtgggcggccaggacgcggcgccggccgacgaggctcccgccgccaaagagatcagcatggtaaacgagaaggtcacccgccgcatcacccccgaagaactccgccagcactcgggcgaggatgagccgtggttcgtcgtcaacggccaggtctatgacggcaccaagttcctcgaaggccatcccggcggcgccgcctccatcatcaacgccgcgggacaagacgtgagcgaggagttcctcgcgattcgtaagttgccctccctccttcctttcttccccccctcccaccccctctcccttcttcccttccaaatttatctcggcaaccaaccctgacacggccaaaaaaaaaaaaaaaaaaacgaaacacagacagcgagaacgccaaggccatgatgccgagttaccacattggcacgctgccggcctcggccgacctctccgaggagccctcggacgaggcggtctcctcctcctccgccgtcttcctccaacccaaaacctggcgccgcgccctcctcgcctccaagacggccgtatcaccggacaccaaggtcttccacttcaccctgccctcgccggcgcaggccgtcggcctcccggtgggccagcacctcctcgtccgcctgcgcgacccggtcacgcgcgaggccatcatccgcgcctacacgccgctgtccgagggcggcgaggtgggcaccctgcgcgtgctcgtcaagatctaccgcgactcgcccgacgggtcgcgcaagggcggccgcatgacgcaggcgctcgactccctgcccgtcggccaccccgtcgagttcaagggccccgtgggcaagttcgagtacctgggcgcggggaggtgcgtcgtcggcggccgggagcggacggtcgcccggttcgtcatggtctgcggcgggtccggcgtgacccccatctttgccgtcctccgggccgtggcgaggggcgagcgggacgagacggcgtgtctggtgctcgacggcaaccgcggggaaggggacatcttgctgcgcgaggaactggatgggctggtgcgggagagccgggggaggtgtaggattgtgcacgctctgagcaggccgagagacgggtggaaggggttgaaggggcggatggatagggccttcttcgagagggaggttggcgctccgccggctgaaaggaacgcgatggtattaatttgcgggcccgaaggcatggagaaggcagcgagggaagcattcttggggatgggctgggatgaggacgatctccttttcttttagtttagcatcatgacatgaactttggaagggcgacgtggcgttggcaggcgtcgggaaaatgcatggtcccggtattcttgtttttgattctaagagggagggggggaagggggagagaaatgtaaagaaaagggcacagccatgattcatgattctcaaatgaccgaccactctggcgaactcgaggggccatgaaagagagtcctcaaggttgaaccgcacatcaaccgaggtggggtgccgtgaaggaagggtgaagtggcgctgcagaactgatttccccggaggtactcaatctttaggcggagacttccagtttagcttgtttattagcccagctgcttgcatctccggcgctgtcagccgagaatggaaggcaccgtgtaacgcagagcacgatttggtgtatttcatccgtataattaactagggaaaagtgagctggaaatacgcaggtactgtacggattaccttcgttgcacaatttaaattctccttagctctgtacagtgaccggtgactctttctggcatgcggagagacggacggacgcagagagaagggctgagtaataagcgccactgcgccagacagctctggcggctctgaggtgcagtggatgattattaatcagggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtctgtttaaacgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacggtttaaac

SEQ ID NO: 20 - DNA, 11788 bp, Artificial sequence
  Synthetic sequence
tcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgggatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgtttcgggtttacctcttccagatacagctcatctgcaatgcattaatgcattggacctcgcaaccctagtacgcccttcaggctccggcgaagcagaagaatagcttagcagagtctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcccctgggttcgcaaagataattgcactgtttcttccttgaactctcaagcctacaggacacacattcatcgtaggtataaacctcgaaaatcattcctactaagatgggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagaaggagaatttaaatcatgacttggttacgaatgagctacgacatgttcaattgagtgactctttactaccaaagtactgctaccatgacactcgaatcgtctcgtgactgaaaggagaatcatgttggcattggttcgcgtagtacggagtaacgacaacggcattggtcaacatctggcaggtatttgaggtagaatataccaacctgcctgaggctctcggtatcaagatttggaaggccaaagggttggatgagcacttgagagcaaagtcggactactggctgaagaaggtaaacaaactaacgtacagtacctacttaacttatgatacacgtcggcgcgcccgcgcgcgacgggcaggtgagaaatcgatgtgtgtctccgtcacaaacctctgcagcagctcgcggccggcgaacatcggtatgtaagttaggtaggttgacaagacgggcatggtgcaggtgtggttggtaggggagatgtctagcctgaagccatacgaagaatgtattgtccatgtgtatgtacaagcgaagcgaatgaaatgcccaaaccgggggcaaaacactgaacgataggtacaacatgaaagaaacaccatgcatcgatgtgtgtgctcgcaagcataccactggtcctcccgagccccaagaagcggtcccgcaaagcgaaggaggaggaggaggaggaagaagaagaagaagaagaagaagaagaagaagtgaaaagagaaatgagaagaagaaaaaaggcccagggccaaacaatgaacaacaagaggaaaacaaaacaaaaaatatgtataacgaatggagagaaaaaagaaaagcggctaataataatgcaaaccatccaagaagagaaaacgaaggtaaacatgtccaggcttcaaaatggtttgccgaatcagtacggcttcatgctccctctctctctctctctctctctctctccagctcccctctctcgaccaggactcgggaggtcatagggccttaattaattacttgccgagggacaggctgagcgacttctgggtgtagtggttgtggagggcctcgtgcatgacggagcagctgaagacgttgccctcttgccagcggctcttgtcgacggtgaggcgggagtacaggaagaacgagccgtccgagtccaggacgggcggggtggtcttgtagttgttctcgggctggccgttggactcccactcgacggcgatgtcgctggggtagaagcccttgacgaggcaggtcagcgagacctggttcttggtcatctcctcttgggagggcgggagggtgtagacctggggctcgcggggctggcccttggccttcgagatggtcttctcgatcgaggaggggaggcccttgttcgagaccttgcacttgtactccttgccgttcagccagtcctggtggaggacggtcaggaccgagacgacgcggtaggtggagttgaactgctcctcgcggggcttggtcttggcgttgtggacctcgacgccgtcgacgtaccagttgaactggacctcggggtcctcttgggagacgtcgacgacgacgcaggtgacctcgggcgtgcgcgagatcatgagggtgtccttgggcttgggcgggaagaggaagacggaggggccgcccaggaactcgggggcggggcagggcgggcagggagggccgtacttgctctcgacgcgcttgtcgaccttggtgttcgagggcttgtggtcgacgttgcaggtgtaggtcttggtgcccaggctggaggaggggacggtgacgacgctcgagagcgagtacaggcccgagctctggaggacggcggggaaggtgtggacgcccgaggtcagggcgcccgagttccacgagacggtgacgggctcggggaagtagtccttgacgaggcagcccagggcggcggtcgactccgaggtgctgcgcgagcagggggccagggggaagacgctggggcccttggtggaggcggaggagacggtgaccagggtgccctggccccagtagtcgtcgttggtggcgcagtagtagacggcggtgtcctcggcgcgcagggagttcatctgcaggaagagggtgttcttggagttgtcgcggctgatggtgaagcggcccttgacggagtcggcgtagtagcgcttgctgccgtcgtaccagatgacggcgacccactcgaggcccttgccgggggcctggcggacccagtgcatgccggagttgctgaaggtgatgcccgaggccttgcagtccaggcggagggagcggccgggctggacgacgccgccgccggactcgaccagctggacctgggcggcggcgccggcgacgagggcggcgagggtggcgaacttggcgtacatggtggacgatggacgattgtctgtgtgagtaagggaaaggtggataggagaaaggggaaggaagttctgaggagggatggaagacggatgatgatgtgctagtaggccccaatgagccgaggagggaaggcctgatctcctttatatccaggaaccatcacggccgtttcccgccatgatctgcccccatctctcccgcgagtttcggaccgtcaacaccgatgaaaaaaagctgggcatcgcttgagagatccgggccctgtgttacgctgagtgcacagcgtgggcgatgtgggcagccggggcctcgacaaggatccaagaacaaccccccaccgcggggtaggttgaccctggcgaaggactggcaaacgaagggccgtgaatggccaacggtcagcaatccggggggctgttgatagtgggcaagggccttgcgcgttgcgatgcacgatattgcggagactgaggagccccactagcgcacccttgggccccgccaacccccaacgggccaaagggccagctctgggagcacggaaggagaacccgaaacacatgtttctggaaagagaaagcttgagcatggtatgatagtgtatttttttcctgcctttcttttctttcccaaccccaagttaggcaggtattcccgtgccttacatgggcacctgggagagactattttggcgcggggggggggtaaacctccgaattcgacctctccttccattgttgattgaattgaagaaggtcagctttcgcagttaccgcgccctcgaaaactcgccagaataatcgcgaaagtcgctccgagtcgctctctccaccttcgcatagtttccggatccacggcccgtgaaggacctgacgagcgctccggctgggtaaacctcggctgattctgggggtgaacaaaccaaaaacacgctggtcacgcgcaggggcgtcgttctagctcagcaaacgaaccacggcaccacgccgcccgcgccgagaggagcatctcgcaagccaggctttctagccaaggcacggggtatccatctctctgaggcgagacgagacgcatccgggccaaggagcactttagcgatgcgcgccctctctggcaaaactccggattttgctttcttgccgtggctaaacctcggtcgctctggcgattcacgtcccttcgctcccaggaaaccgaaccgtgcggccgactctgaacgagccgggcggctctcttcttcttttctcttctttcttctcgtcccatacgcttacttggaacactatgggcgacggcaactcgggctggctgttccggagcatggacatcgccgctctgcttatcgagtgtttggtgtgtcagacccgctccatcattccgtgaccgtggcaacccgccggcgacgaacctctctgaaggaggttctgagacacgcgcgattcttctgtatatagttttatttttcactctggagtgcttcgctccaccagtacataaaccttttttttcacgtaacaaaatggcttcttttcagaccatgtgaaccatcttgatgccttgacctcttcagttctcactttaacgtagttcgcgtttgtctgtatgtcccagttgcatgtagttgagataaatacccctggaagtgggtctgggcctttgtgggacggagccctctttctgtggtctggagagcccgctctctaccgcctaccttcttaccacagtacactactcacacattgctgaactgacccatcataccgtactttatcctgttaattcgtggtgctgtcgactattctatttgctcaaatggagagcacattcatcggcgcagggatacacggtttatggaccccaagagtgtaaggactattattagtaatattatatgcctctaggcgccttaacttcaacaggcgagcactactaatcaacttttggtagacccaattacaaacgaccatacgtgccggaaattttgggattccgtccgctctccccaaccaagctagaagaggcaacgaacagccaatcccggtgctaattaaattatatggttcattttttttaaaaaaattttttcttcccattttcctctcgcttttctttttcgcatcgtagttgatcaaagtccaagtcaagcgagctatttgtgcgtttaaacgctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacggtttaaac

SEQ ID NO: 21 - DNA, 18 bp, Artificial sequence
  Synthetic sequence
atcagaccacgacgggac

SEQ ID NO: 22 - DNA, 21 bp, Artificial sequence
  Synthetic sequence
caggtaggaggagaccgactg

SEQ ID NO: 23 - DNA, 20 bp, Artificial sequence
  Synthetic sequence
tagcgcgaatactgctgtgg

SEQ ID NO: 24 - DNA, 19 bp, Artificial sequence
  Synthetic sequence
catgccggagttgctgaag

SEQ ID NO: 25 - DNA, 19 bp, Artificial sequence
  Synthetic sequence
gctgacgcgaatgacacag

SEQ ID NO: 26 - DNA, 19 bp, Artificial sequence
  Synthetic sequence
catgcccttgccgtagaag

SEQ ID NO: 27 - protein, 750 aa, Myceliophthora thermophila
MSADPLQVLASVGKGKSTRGVLRVIILALIAAAAIASRLFSVIRFESIIHEFDPWFNFRATKYLVANGFYNFWDWFDDRTWHPLGRVTGGTLYPGLMVTSGVIYHALRALTIPVDIRNICVLLAPGFSGLTAIAAYLLTNEMTPSPSAGLLAALFMGIAPGYISRSVAGSYDNEAIAIFLLVFTFYLWIKALKQGSMLWGALCALSYGYMVASWGGYAFITCLVPLHSFVLICMGRYSTRLYVAYTTWYALGTLASMQIPFVGFLPVKTSEHMPALGIFGFLQLLGFLDYVRSAIPSRQFQTFLWLFAGATFGLGLLGLVIATSAGFIAPWSGRFYSLWDTGYAKIHIPIIASVSEHQPTAWPAFFFDLNLLVWLFPVGVYLCFQQLADEHVFIVVYALFGSYFAGVMVRLMLTLTPVVCVAAAIAVSTLLDTYLSTKSLKPQESQATEGPESKKAAKSSGLKSTSKPVVGVYALWGKSMMVGALAVYLLLFVLHCTWVTSNAYSSPSVVLASRLPDGSQHIIDDYREAYQWLRQNTREDAKIMSWWDYGYQIGGMADRPTLVDNNTWNNTHIATVGKAMSSREEVSYPIMRQHEVDYVLVVFGGLLGYSGDDINKFLWMVRIAEGIWPDEVNERSFFTARGEYRVDGEATETMKNSLMYKMSYYNYNNLFPPGQAVDRMRGARLPEVGPTLSTLEEAFTSENWIIRIYKVKDLDNLGRDHMSAAAFDRGHKKKSKGITKKKGPRQLRID

SEQ ID NO: 28 - protein, 309 aa, Myceliophthora thermophila
MAADDDITPLASLSLTHVYYNPTDPLSTASAFLSLLPQALCVVYATLLWSTREAEVLLMFLGQLACEAINFALKRLIKEERPPRLGVGKGYGMPSSHAQFAAFWAVALALFLCVRHRPARLGPPRRPPRLRLGGRAVVPPGKGDRAAATVADGNRNGNGNGDAEPRLSDVHRAYVSGGLAAASRSIEAYSHSHWTMAQRVVVSAGALLVAGLVAWSRVYLGYHTVRQVVAGSAAGAGCAAAWFAVTYLMRETGMVAWALELPPARWFRMRDLVVEEDLCQAGWEKWQEKRQLALEREQSRERVREKKGQ

SEQ ID NO: 29 - DNA, 8017 bp, Myceliophthora thermophila
agggtaggtgggatgggcggggtgtagggtaggtcggtgtagggtaggtcggctgggcggggtgtagggtaggtcggttgggcggggtgtagggtaggtcggttgggatgggtgtagggtaggtcggccgggtgtagggtaggtcggctgggcggggtgtagggtaggtcggtgtagggtaggtgggatggggcgctatgtgcggccgcgagctcgcgagcccatttttagcgaaggccatacaaacgagttttgcggaacccgggattccacccccgaagccgccggcgcgtgcgccccgctgcgcatcggtcggtgggtatatgagaagggggcgggcaagccggaagccagaggcaactgctactgttagctgccgctggcctccgcggcccagggcgcggcacggctgcgttgaagtctcccagtctcccacccgttggctgcgcggatccgcccgtcttggtggttgcgagctcgcgagcccatttttagcgaaggccatacaaacgagttttgcggggcccgggattccaccccggaacccgccggcgcgtgcgccccgctgcgcatcggtcggtgggtatgtgagggaggaagaagaaaaaaaaaaaaagctcctgcgggggggctgtcgggcacgcctactttcgggcgacccggcacctctccgcggcagccttcgcaggccgctgttggtcccatttcatacgtcgccgccttcgcgtggtgccctacggtctgccggggtaccgacgattgcggcgagcaccgcctcagcaccgctgctgccaccggcgcgacctcgcccgggggtgcgcgcggcatctgggaagactctgcaggcgtaagggaataccccatgtgcgccgaggggtgggctatgtgggtgcttggcggttcgccagacctttctaaagccaccgggggtacctaccggttggggacgcctacagggctgaacctcccggtcgggcctcctcttggggcgcttaggcggcgacttcggggcgcgatcgctccccgctctcgcccgccgacggcgctctggggaattcaggaggggaaagcagatgtgacccgcggctcgaccggcgcattgccggacgagctgcgcggccacgcgggcccccgcgcccgccgacccagtaacttagtgaactcttccgccctgaaacacgggcggttggccctaaccggctcacgatagttacctggttgattctgccagtagtcatatgcttgtctcaaagattaagccatgcatgtctaagtataagcaattatacagcgaaactgcgaatggctcattaaatcagttatcgtttatttgatagtaccttactacatggataaccgtggtaattctagagctaatacatgctaaaaatcccgacttcggaagggatgtatttattagattaaaaaccaatgccctccggggctctctggtgattcatgataacttctcgaatcgcacggccttgcgccggcgatggttcattcaaatttctgccctatcaactttcgacggctgggtcttggccagccgtggtgacaacgggtaacggagggttagggctcgaccccggagaaggagcctgagaaacggctactacatccaaggaaggcagcaggcgcgcaaattacccaatcccgacacggggaggtagtgacaataaatactgatacagggctcttttgggtcttgtaattggaatgagtacaatttaaatcccttaacgaggaacaattggagggcaagtctggtgccagcagccgcggtaattccagctccaatagcgtatattaaagttgttgaggttaaaaagctcgtagttgaaccttgggcctagccggccggtccgcctcaccgcgtgcactggctcggctgggtctttccttctggagaaccgcatgcccttcactgggtgtgccggggaaccaggacttttactctgaacaaattagatcgcttaaagaaggcctatgctcgaatacattagcatggaataatagaataggacgtgtggttctattttgttggtttctaggaccgccgtaatgattaatagggacagtcgggggcatcagtattcaattgtcagaggtgaaattcttggatttattgaagactaactactgcgaaagcatttgccaaggatgttttcattaatcaggaacgaaagttaggggatcgaagacgatcagataccgtcgtagtcttaaccataaactatgccgattagggatcggacggcgttattttttgacccgttcggcaccttacgataaatcaaaatgtttgggctcctgggggagtatggtcgcaaggctgaaacttaaagaaattgacggaagggcaccaccaggggtggagcctgcggcttaatttgactcaacacggggaaactcaccaggtccagacacgatgaggattgacagattgagagctctttcttgatttcgtgggtggtggtgcatggccgttcttagttggtggagtgatttgtctgcttaattgcgataacgaacgagaccttaacctgctaaatagcccgtattgctttggcagtacgccggcttcttagagggactatcggctcaagccgatggaagtttgaggcaataacaggtctgtgatgcccttagatgttctgggccgcacgcgcgctacactgacagagccagcgagtactcccttggccggaaggcccgggtaatcttgttaaactctgtcgtgctggggatagagcattgcaattattgctcttcaacgaggaatccctagtaagcgcaagtcatcagcttgcgttgattacgtccctgccctttgtacacaccgcccgtcgctactaccgattgaatggctcagtgaggctttcggactggcccagagaggtcggcaacgaccactcagggccggaaagttatccaaactcggtcatttagaggaagtaaaagtcgtaacaaggtctccgttggtgaaccagcggagggatcattacagagctgcaaaactccctaaaccatcgtgaacgctacctagaccgttgcttcggcgggcggcgccctcgcgcgccccccctggggcccgcaccgcgggcgcccgccggaggtacaccaaactcttgatatgttatggccactctgagtctcctgtactgaataagtcaaaactttcaacaacggatctcttggttctggcatcgatgaagaacgcagcgaaatgcgataagtaatgtgaattgcagaattcagtgaatcatcgaatctttgaacgcacattgcgcccgccagcatcctggcgggcatgcctgttcgagcgtcatttcaacccatcaagcccacggcttgtgttggggacctgcggctgcccgcaggccctgaaaaccagtggcgggctcgctagtcacaccgggcgtagtagcatacgacctcgctcagggcgtgctgcgggttccagccgtaaaacgaccttcacaacccaaggttgacctcggatcaggtaggaggacccgctgaacttaagcatatcaataagcggaggaaaagaaaccaacagggattgccctagtaacggcgagtgaagcggcaacagctcaaatttgaaatctggcttcggcccgagttgtaatttgcagaggaagctttaggcgcggcaccttctgagtcccctggaacggggcgccatagagggtgagagccccgtatagttggatgcctagcctgtgtaaagctccttcgacgagtcgagtagtttgggaatgctgctcaaaatgggaggtaaatttcttctaaagctaaataccggccagagaccgatagcgcacaagtagagtgatcgaaagatgaaaagcactttgaaaagagggttaaatagcacgtgaaattgttgaaagggaagcgcttgtgaccagacttgcgccgggctgatcatccggtgttctcaccggtgcactctgcccggctcaggccagcatcggttctcgcggggggataaaggcccggggaatgtagctcctccgggagtgttatagccccgggtgtaataccctcgcggggaccgaggttcgcgcatctgcaaggatgctggcgtaatggtcatcagcgacccgtcttgaaacacggaccaaggagtcaaggttttgcgcgagtgtttgggtgtaaaacccgcacgcgtaatgaaagtgaacgtaggtgagagcttcggcgcatcatcgaccgatcctgatgttttcggatggatttgagtaggagcgttaagccttggacccgaaagatggtgaactatgcttggatagggtgaagccagaggaaactctggtggaggctcgcagcggttctgacgtgcaaatcgatcgtcaaatctgagcatgggggcgaaagactaatcgaaccatctagtagctggttaccgccgaagtttccctcaggatagcagtgttgtcttcagttttatgaggtaaagcgaatgattagggactcgggggcgctttttagccttcatccattctcaaactttaaatatgtaagaagcccttgttacttagttgaacgtgggccttcgaatgtatcaacactagtgggccatttttggtaagcagaactggcgatgcgggatgaaccgaacgcggggttaaggtgccggagtggacgctcatcagacaccacaaaaggcgttagtacatcttgacagcaggacggtggccatggaagtcggaatccgctaaggactgtgtaacaactcacctgccgaatgtactagccctgaaaatggatggcgctcaagcgtcccacccataccccgccctcagggtagaaacgacgccctgaggagtaggcggccgtggaggtcagtgacgaagcctagggcgtgagcccgggtcgaacggcctctagtgcagatcttggtggtagtagcaaatacttcaatgagaacttgaaggaccgaagtggggaaaggttccatgtgaacagcggttggacatgggttagtcgatcctaagccatagggaagttccgtttcaaaggggcactcgtgccccgtgtggcgaaagggaagccggttaacattccggcacctggatgtgggttttgcgcggtaacgcaactgaacgcggagacgacggcgggggccccgggcagagttctcttttcttcttaacggtctatcaccctggaaacagtttgtctggagatagggtttaacggccggaagagcccgacacttctgtcgggtccggtgcgctctcgacgtcccttgaaaatccgcgggagggaataattctcacgccaggtcgtactcataaccgcagcaggtccccaaggtgaacagcctctggttgatagaacaatgtagataagggaagtcggcaaaatagatccgtaacttcgggataaggattggctctaagggttgggcacgttgggctttgggcggacgccctgggagcaggtcgcctctagccgggcaaccggcggggggcttccagcatccgggtgcagatgcccttagcaggcttcggccgtccggcgcgcggttaacaaccaacttagaactggtacggacagggggaatctgactgtctaattaaaacatagcattgcgatggccagaaagtggtgttgacgcaatgtgatttctgcccagtgctctgaatgtcaaagtgaagaaattcaaccaagcgcgggtaaacggcgggagtaactatgactctcttaaggtagccaaatgcctcgtcatctaattagtgacgcgcatgaatggattaacgagattcccactgtccctatctactatctagcgaaaccacagccaagggaacgggcttggcagaatcagcggggaaagaagaccctgttgagcttgactctagtttgacattgtgaaaagacataggaggtgtagaataggtgggagcttcggcgccggtgaaataccactactcctattgtttttttacttattcaatgaagcggggctggattttcgtccaacttctggttttaaggtccttcgcgggccgacccgggttgaagacattgtcaggtggggagtttggctggggcggcacatctgttaaaccataacgcaggtgtcctaaggggggctcatggagaacagaaatctccagtagaacaaaagggtaaaagtccccttgattttgattttcagtgtgaatacaaaccatgaaagtgtggcctatcgatcctttagtccctcgaaatttgaggctagaggtgccagaaaagttaccacagggataactggcttgtggcggccaagcgttcatagcgacgtcgctttttgatccttcgatgtcggctcttcctatcataccgaagcagaattcggtaagcgttggattgttcacccactaatagggaacgtgagctgggtttagaccgtcgtgagacaggttagttttaccctactgatgaactcatcgcaatggtaattcagcttagtacgagaggaaccgctgattcagataattggtttttgcggttgtccgaccgggcagtgccgcgaagctaccatctgctggataatggctgaacgcctctaagtcagaatccatgccagaacgcgatgatactacccgcacgttgtagacgtataagaataggctccggcctcgtatcctagcaggcgattcctccgccggcctcgaagttggccggcggtaattcgcgtattgcaatttcgacacgcgcgggatcaaatcctttgcagacgacttagatgtgcgaaagggtcctgtaagcagtagagtagccttgttgttacgatctgctgagggtaagccctccttcgcctagatttcccagcgagagcccgccggcggaacagccgggcgagccttacgggggaagccttaaggggattgagaagtggtgccgtgcgttcgcgcgcccctaggtcctttagccggccgcaggtgtagggtaggtcggttgggaggatggggtgtagggtaggtcggtgtagggtaggttggttgggaggatggggtgtagggtaggtcggccgggtgtagggtaggtcggtgtagggtaggtgggatggggcgctatatgcggccgcgagctcgcgagcctatttttagtgaaggctatataaataagctttacgttaccgggccttgctaccctcgagtggcgtgggccgtgctgcctactgggcattgctcgccgggctgtataagggaggggtcggggtcgcggtctagggtaggtcgggtgggatggggtgtagggtaggagaagcgctctagtcgtgtgtctttttctctaggtctattattagtactggctgtagggcgacgtgccctgccttgttataatattatattgtatgtttaggcctatactagcttgtaatctatttgtatctggcttattaggtacggcttcctttgtatataactagagaggctctggtatgcttcttagtatagcggtataggattcataatcatagtaatgataatcataatagtaataataataataatagtaatgataataataataatctatttatatcttatttaaaatgcttgtacggctgcctgctcttaaggagtagctagatatgagatggtagggtagctagctaacctaggctagacgttctcgtcccttagctatataagtgctatatattatagttagttatctaacctaccttcttacttgagcagaagaggtagggttctagtatagctagtagggcttctaggcctaagggcctgttattcgagttattataggttagtatttaatatagttatagggataggcctcgattacgggtataggataggtaggataggtatagggtaggtcggttaggaggatagggtgtaaggtaggtcggccgggtatagggtaggtagtaggttaggcggggtgtagggtaggtcggtgtagggtaggtgggatgggcggggtgtagggtaggtcggttgggaggatggggtgtagggtaggtcggtgtagggtaggtcgg